SEQUENCE LISTING (16 sequences)

SEQ ID NO: 1 - DNA, 354 bp, Mus musculus
gaggttcacttacagcagtctggggcagagcttgtgaagcctggggcctcagtcaggttgtcctgcacaggttctggcttagacattaaagacacctatattcactgggtgaagaagaggcctgaacagggcctggagtggattggaaggattgatcctgcgaatggtattactaaatatgacccgaagttccagggcaaggccactgtaacagcagacacatcctccaacacagcctacctgcagttcagcagcctcacatcagaggacagtgccgtctactactgttcgagtgagtactatccttatcctatggactactggggtcaaggaacctcagtcaccgtctcctca

SEQ ID NO: 2 - protein, 118 aa, Mus musculus
EVHLQQSGAELVKPGASVRLSCTGSGLDIKDTYIHWVKKRPEQGLEWIGRIDPANGITKYDPKFQGKATVTADTSSNTAYLQFSSLTSEDSAVYYCSSEYYPYPMDYWGQGTSVTVSS

SEQ ID NO: 3 - protein, 8 aa, Mus musculus
GLDIKDTY

SEQ ID NO: 4 - protein, 8 aa, Mus musculus
IDPANGIT

SEQ ID NO: 5 - protein, 11 aa, Mus musculus
SSEYYPYPMDY

SEQ ID NO: 6 - DNA, 321 bp, Mus musculus
gacatcaagatgacccagcctccatcttccatgtatgcatctctaggagagagagtcactgtcacttgtaaggcgagtcaggacattagtagttttttaacctggttccaacagaaaccagggaaatctcctaagatcctgatctatcgtgcaaacagagtggtagatggggtcccatcaaggttcagtggcagtggatctggccacgattattctctcaccatcagcagcctggagtctgaagatatgggaatttattattgtctacaatatgatgagtttccgtggacgttcggtggaggcaccaagctggagatcaaa

SEQ ID NO: 7 - protein, 107 aa, Mus musculus
DIKMTQPPSSMYASLGERVTVTCKASQDISSFLTWFQQKPGKSPKILIYRANRVVDGVPSRFSGSGSGHDYSLTISSLESEDMGIYYCLQYDEFPWTFGGGTKLEIK

SEQ ID NO: 8 - protein, 6 aa, Mus musculus
QDISSF

SEQ ID NO: 9 - protein, 3 aa, Mus musculus
RAN

SEQ ID NO: 10 - protein, 9 aa, Mus musculus
LQYDEFPWT

SEQ ID NO: 11 - DNA, 24 bp, Mus musculus
ggcttagacattaaagacacctat

SEQ ID NO: 12 - DNA, 24 bp, Mus musculus
attgatcctgcgaatggtattact

SEQ ID NO: 13 - DNA, 33 bp, Mus musculus
tcgagtgagtactatccttatcctatggactac

SEQ ID NO: 14 - DNA, 18 bp, Mus musculus
caggacattagtagtttt

SEQ ID NO: 15 - DNA, 9 bp, Mus musculus
cgtgcaaac

SEQ ID NO: 16 - DNA, 27 bp, Mus musculus
ctacaatatgatgagtttccgtggacg

(19) United States
(12) Patent Application Publication
(10) Pub. No.: US 2014/0286960 A1
(43) Pub. Date: Sep. 25, 2014

(54) STAPHLOCOCCUS AUREUS ALPHA-HEMOLYSIN ANTIBODIES

(71) Applicants: Jody Berry, San Diego, CA (US); Laura Saward, Winnipeg (CA); Robyn Cassan, Winnipeg (CA); Xiaobing Han, Winnipeg (CA); Mohammad Javad Aman, Rockville, MD (US); Rajan P. Adhikari, Gaithersburg, MD (US); Hatice Karauzum, Silverspring, MD (US)

(72) Inventors: Jody Berry, San Diego, CA (US); Laura Saward, Winnipeg (CA); Robyn Cassan, Winnipeg (CA); Xiaobing Han, Winnipeg (CA); Mohammad Javad Aman, Rockville, MD (US); Rajan P. Adhikari, Gaithersburg, MD (US); Hatice Karauzum, Silverspring, MD (US)

(73) Assignee: Cangene Corporation, Winnipeg (CA)

(21) Appl. No.: 14/235,315

(22) PCT Filed: Jul. 27, 2012
(86) PCT No.: PCT/CA2012/050515

§ 371 Date: May 27, 2014

Related U.S. Application Data
(60) Provisional application No. 61/512,518, filed on Jul. 28, 2011.

(51) Int. Cl. A61K 39/40; A61K 45/06; C07K 16/12

(52) U.S. Cl. CPC A61K 39/40; C07K 16/1271; A61K 45/06
USPC 424/150.1; 530/388.4; 536/23.53; 435/320.1; 435/340

(57) ABSTRACT

Compositions and methods for the treatment or prevention of Staphyloccus aureus infection in a subject are provided. Antibody compositions comprising monoclonal antibodies to alpha-hemolysin protein are also provided. The methods provide administering a composition to the subject in an amount effective to reduce or eliminate or prevent relapse S. aureus bacterial infection and/or induce an immune response to Staphylococcus aureus alpha-hemolysin.

### RELATED APPLICATIONS

This patent claims priority to U.S. provisional patent application 61/512,518, filed on Jul. 28, 2011 and incorporated herein by reference.

### FIELD OF THE INVENTION

The invention relates to monoclonal antibodies to Staphylococcus aureus alpha-hemolysin. The invention further relates to compositions and methods for the treatment or prevention of infection by the bacteria, Staphylococcus aureus, in a vertebrate subject. Methods are provided for administering antibodies to the vertebrate subject in an amount effective to reduce, eliminate, or prevent relapse from infection. Methods for the treatment or prevention of Staphylococcus aureus infection in an organism are provided.

### BACKGROUND OF THE INVENTION

Staphylococcus aureus (S. aureus) is a ubiquitous gram positive bacterium that can colonize the nares and skin of humans without causing disease. Approximately one third of the human population is colonized with S. aureus making it difficult to avoid transmittance. The bacteria can cause a wide variety of disease from mild skin infections to more serious diseases such as bacteremia and endocarditis. The patient populations most at risk are dialysis patients, patients with ventriculoperitoneal shunts, patients at risk of infective endocarditis, patients who are immunocompromised, and residents of nursing homes. In healthcare settings it is the main pathogen responsible for infections of the skin and soft tissues, as well as for those associated with medical procedures and indwelling devices such as catheters. Since catheter- and device-related infections remain the most significant cause of morbidity, prolonged length of stay and increased cost in affected patients, S. aureus infections are of concern. S. aureus has developed resistance to multiple antibiotics and has a methicillin-resistant variant (MRSA) which is becoming widespread in the community and nosocomial environments. This is leading to increased incidences of infection in both the hospital and community settings. With reduced treatment options available, alternative approaches are required.

S. aureus alpha-hemolysin (Hla) is a self-assembling, pore-forming β-barrel with cytotoxic properties. Hla is known to play an important role in the pathogenesis of S. aureus infection. S. aureus mutants lacking hla are less virulent in animal models of intraperitoneal, intranasal and intramammary infections (Bramley et al., 1989; Bubeck et al., 2007; Patel et al., 1987). Active immunization with a mutant form of Hla (Hla_{H35L}), which can not form pores, generates antigen-specific IgG response and provide a high degree of protection against S. aureus infections (Bubeck et al., 2008). Moreover, passive immunization of mice with anti-Hla antisera or monoclonal antibodies provides protection against both toxin challenge and live S. aureus infection (Menzies et al., 1996; Kennedy et al., 2010; Ragle et al., 2008). Hla is secreted by the vast majority of clinical S. aureus isolates and is highly conserved (Kobayashi et al, 2009). A recent study using transposon insertions indentified 72 genes that affect alpha-hemolysin expression in S. aureus (Burnside et al., 2010). When virulence determinants were studied for S. aureus epidemic strains, Hla was found to be produced in higher level in virulent strains, such as USA300, compared to other strains, which contributes to the high virulence of community-associated methicillin-resistant S. aureus (CA-MRSA) infection (Li et al., 2010).

Biofilms are surface-associated, sessile bacterial communities which are formed when planktonic cells colonize a surface embedded in an exopolysaccharide matrix, such as a catheter, followed by aggregation and growth into multi-cellular colonies. S. aureus has the capability to form biofilms on surfaces such as intravascular catheters and pacemaker leads which increases its persistence and boosts its antimicrobial resistance making it difficult to clear the infection. The mechanisms of antibiotic tolerance in biofilms are thought to be due to altered metabolic activity, diffusion limitations, and differences in the genotypes and phenotypes of biofilm cells compared to planktonic bacteria. It is thought that blocking the colonization of the bacteria rather than protecting against infection might be more achievable and effective. IgG antibodies against cell wall-associated MRSA proteins were shown to penetrate S. epidermidis biofilms leading to the hypothesis that antibodies to specific biofilm-upregulated, cell wall-associated antigens could aid in blocking colonization and break the cell-cell interactions of the biofilm.

Hla has been showed to play an integral role in S. aureus biofilm formation. The study showed that the hla mutant is capable of initially colonizing a surface but never organizes into multicellular macrocolonies, indicating a defeat in cell-to-cell interaction in mutant strain (Caiazza et al., 2003). Biofilm development is thought to consist of two steps: the initial cell-to-surface interactions and the subsequent cell-to-cell interactions. The accessory gene regulator (agr) is a two-component regulatory system in S. aureus that has been implicated in biofilm formation. One of the downstream targets regulated by the agr system is Hla, which causes host cell lysis by heptamerizing upon insertion into eukaryotic cell membranes in addition to playing a role in biofilm formation. Mutants defective in Hla production failed to form biofilms under both static and flow conditions, and strains lacking Hla have an apparent defect in cell-to-cell interactions (O'Toole G A, et al. J Bac, 2003).

Attempts have been made and are ongoing to develop a vaccine for S. aureus, however, most have failed at various stages in clinical trials. This is likely due to the pathogenosis of the bacteria which is multi-factorial with a large number of virulence factors. Other issues include the broad spectrum of different diseases it causes, the complex regulatory pathways that govern the expression of surface antigens can differ from strain to strain, and its ability to evade the immune system. Due to the large number of virulence factors, the bacteria could be adept at avoiding the effects of the vaccine developed to a specific target simply by altering expression of the vaccine target. Therefore, there is an unmet need for effective treatment and/or prevention of S. aureus associated infections.

### SUMMARY OF THE INVENTION

Described herein are antibodies, compositions and methods for the treatment of Staphylococcus infections in vertebrates.

Described herein are antibodies, compositions and methods for the treatment of S. aureus infection in vertebrates.

In a first aspect, the present invention provides compositions comprising an antibody or fragment thereof that binds to S. aureus alpha-hemolysin or fragment thereof.

In a further aspect, the present invention provides an isolated antibody or fragment thereof that binds to S. aureus alpha-hemolysin or fragment thereof.

In a further aspect, the antibody or fragment thereof is a humanized antibody.

In a further aspect, the antibody or fragment thereof has high affinity, high binding specificity, or both high affinity and high specificity, to S. aureus alpha-hemolysin protein.

In a further aspect of the present invention the isolated antibody is CAN24G4 or an active fragment thereof.

In yet a further aspect of the present invention, the isolated antibody is CAN24G5 or an active fragment thereof.

In yet a further aspect of the present invention is an antibody or an antibody fragment having the affinity, the binding specificity, or both the affinity and the binding specificity of CAN 24G4.

In yet a further aspect of the present invention is an antibody or an antibody fragment having the affinity, the binding specificity, or both the affinity and the binding specificity of CAN 24G5.

In a further aspect of the present invention, the antibody or fragment thereof can be: a monoclonal antibody; a murine antibody; a human antibody; a whole immunoglobulin antibody; an scFv; a chimeric antibody; a Fab fragment; an F(ab′)2; a bispecific antibody construct; or a disulfide linked Fv.

In a further aspect of the present invention, the antibody is a humanized antibody.

In other embodiments of the invention the antibody or fragment thereof can have a heavy chain immunoglobulin constant domain, which can be a human IgM constant domain; a human IgG1 constant domain, a human IgG2 constant domain, a human IgG3 constant domain, a human IgG4 constant domain, or a human IgA1/2 constant domain.

In other embodiments of the present invention the antibody or fragment thereof can have a light chain immunoglobulin constant domain, which can be a human Ig kappa constant domain or a human Ig lambda constant domain.

In yet a further aspect of the present invention, the antibody or fragment thereof has the epitope binding characteristics of CAN24G4 antibody. In certain embodiments, the antibody or fragment thereof selectively and specifically binds to an epitope of alpha-hemolysin identical to that which binds to CAN24G4

In yet a further aspect of the present invention, the antibody or fragment thereof has the epitope binding characteristics of CAN 24G5 antibody. In certain embodiments, the antibody or fragment thereof selectively and specifically binds to an epitope of alpha-hemolysin identical to that which binds to CAN24G5.

A further aspect of the present invention is a pharmaceutical composition comprising the antibody as herein described, for example, CAN 24G4 antibody, CAN 24G5 antibody, both CAN 24G4 and CAN 24G5 antibodies, and/or an active fragment thereof. The pharmaceutical composition may include a pharmaceutically acceptable carrier. The pharmaceutical composition can be formulated for intravenous, subcutaneous, intramuscular, or oral administration.

In another aspect of the invention, the antibody or fragment thereof has a neutralizing effect on S. Aureus alpha-hemolysin protein. In certain embodiments, this neutralizing effect is through the interruption of the biological activity of S. Aureus alpha-hemolysin protein. In certain aspects, the antibody or fragment thereof has high binding specificity to S. Aureus alpha-hemolysin protein.

In certain embodiments, the pharmaceutical composition comprises CAN 24G4 and CAN 24G5 in a 1:1 (activity:activity) ratio.

In certain embodiments, the pharmaceutical composition comprises CAN 24G4 and CAN 24G5 in a 1:1 (concentration) ratio.

In certain embodiments, the antibody or fragment thereof is humanized.

In certain embodiments, the pharmaceutical composition can further comprise a pharmaceutically acceptable adjuvant, for example, an oil-in-water emulsion, ISA-206, Quil A, interleukin 12 and a heat shock protein.

In certain embodiments, the pharmaceutical composition can further comprise an antibiotic.

In a further aspect of the present invention, the composition can be used in a method of treatment of S. aureus associated disease by administration to a subject in need of such treatment an amount of the composition effective to reduce or prevent the disease, which can be for example an amount in the range of 1 to 100 milligrams per kilogram of the subject's body weight. The compositions can be administered intravenously (IV), subcutaneously (SC), intramuscularly (IM), transdermally or orally. The method may be to decrease morbidity in the subject, to prevent or treat bacteremia in a subject, and/or to prevent or treat dermal necrosis in a subject. The method may be used to treat or prevent biofilm formation.

In another aspect of the present invention, the compositions can be used in a method of passive immunization by administration to an animal of an effective amount of the composition.

A further aspect of the present invention provides a method of treating or preventing biofilm formation in a subject in need of such treatment by administering to the subject S. aureus alpha-hemolysin antibodies, or functionally active variants or fragments thereof.

A further aspect of the present invention is an S. aureus alpha-hemolysin antibody, or functionally active variant or fragment thereof. In certain embodiments the antibody or functionally active variant or fragment thereof has high affinity to alpha-hemolysin. In certain embodiments the antibody, or functionally active variant or fragment thereof, has high specificity to alpha-hemolysin. In certain embodiments the antibody, or functionally active variant or fragment thereof is a monoclonal antibody. In certain embodiments the antibody, or functionally active variant or fragment thereof is a humanized monoclonal antibody.

A further aspect of the present invention is an S. aureus alpha-hemolysin antibody known as Can24G4 antibody. A further aspect of the present invention is an S. aureus alpha-hemolysin antibody known as Can24G5 antibody.

A further aspect of the present invention is a fragment of Can24G4 that selectively binds S. aureus alpha-hemolysin. A further aspect of the present invention is a fragment of Can24G5 that binds S. aureus alpha-hemolysin.

In certain embodiments the monoclonal antibody comprises a heavy chain variable region having an amino acid sequence translated from a nucleotide sequence 80%, preferably 90%, more preferably 95%, most preferably 100%, identical to the nucleotide sequence of SEQ ID NO. 1. In certain embodiments the monoclonal antibody comprises two heavy chain variable regions each having an amino acid sequence translated from said nucleotide sequence. In certain embodiments, the monoclonal antibody comprises a light chain variable region having an amino acid sequence translated from a nucleotide sequence 80%, preferably 90%, more preferably 95%, most preferably 100%, identical to the nucleotide sequence of SEQ ID NO. 6. In certain embodiments, the monoclonal antibody comprises two light chain variable regions each having an amino acid sequence translated from said nucleotide sequence. In certain embodiments, the monoclonal antibody comprises a light chain variable region having an amino acid sequence translated from the nucleotide sequence of SEQ ID NO. 6 and a heavy chain variable region having an amino acid sequence translated from the nucleotide sequence of SEQ ID NO. 1. In certain embodiments, the monoclonal antibody consists of two light chains, each having a variable region having an amino acid sequence translated from the nucleotide sequence of SEQ ID NO. 6 and two heavy chains, each having a variable region having an amino acid sequence translated from the nucleotide sequence of SEQ ID NO. 6.

In certain embodiments, the monoclonal antibody comprises a heavy chain variable region having an amino acid sequence 80%, preferably 90%, more preferably 95%, most preferably 100% identical to the amino acid sequence of SEQ ID NO. 2. In certain embodiments, the monoclonal antibody comprises two heavy chain variable regions each having said amino acid sequence. In certain embodiments, the monoclonal antibody comprises a light chain variable region having an amino acid sequence of SEQ ID NO. 7. In certain embodiments, the monoclonal antibody comprises two light chains each having variable regions having an amino acid sequence of SEQ ID NO. 7. In certain embodiments, the monoclonal antibody comprises a light chain having a variable region having an amino acid sequence of SEQ ID NO. 7 and a heavy chain having a variable region having an amino acid sequence of SEQ ID NO. 2. In certain embodiments, the monoclonal antibody consists of two light chains each having a variable region having an amino acid sequence of SEQ ID NO. 7 and two heavy chains each having a variable region having an amino acid sequence of SEQ ID NO. 2.

In certain embodiments, the monoclonal antibody has at least one CDR region selected from the group consisting of SEQ ID NO. 3, SEQ ID NO. 4, SEQ ID NO. 5, SEQ ID NO. 8, SEQ ID NO. 9, and SEQ ID NO. 10. In certain embodiments, the monoclonal antibody has at least two CDR regions selected from the group consisting of SEQ ID NO. 3, SEQ ID NO. 4, SEQ ID NO. 5, SEQ ID NO. 8, SEQ ID NO. 9, and SEQ ID NO. 10. In certain embodiments, the monoclonal antibody has at least three CDR regions selected from the group consisting of SEQ ID NO. 3, SEQ ID NO. 4, SEQ ID NO. 5, SEQ ID NO. 8, SEQ ID NO. 9, and SEQ ID NO. 10. In certain embodiments, the monoclonal antibody has at least four CDR regions selected from the group consisting of SEQ ID NO. 3, SEQ ID NO. 4, SEQ ID NO. 5, SEQ ID NO. 8, SEQ ID NO. 9, and SEQ ID NO. 10. In certain embodiments, the monoclonal antibody has at least five CDR selected from the group consisting of SEQ ID NO. 3, SEQ ID NO. 4, SEQ ID NO. 5, SEQ ID NO. 8, SEQ ID NO. 9, and SEQ ID NO. 10. In certain embodiments, the monoclonal antibody has the following six CDR regions: SEQ ID NOs. 3, 4, 5, 8, 9, and 10.

In certain embodiments, the antibody is a monoclonal antibody. In other embodiments, the antibody is a polyclonal antibody.

A further embodiment of the invention is the use of the antibody or fragment as hereindescribed, or the pharmaceutical composition as hereindescribed, in the preparation of a medicament. The medicament may be for the reduction or prevention of S. aureus infection, may be for the reduction of morbidity, may be for passive immunization to S. aureus infection, may be for treatment or prevention of bacteremia, and/or may be for treatment or prevention of dermal necrosis.

Yet a further embodiment of the invention is the use of the antibody or fragment as hereindescribed, or the pharmaceutical composition as hereindescribed, for the reduction or prevention of S. aureus infection, for the reduction of morbidity, for passive immunization to S. aureus infection, for the treatment or prevention of bacteremia, and/or for the prevention or treatment of dermal necrosis.

According to a further aspect of the invention is provided an isolated nucleic acid encoding an antibody or fragment thereof that selectively binds to S. aureus alpha-hemolysin protein. The isolated nucleic acid may have a nucleotide sequence having at least 80%, preferably at least 90%, more preferably at least 95%, or 100% identity with a nucleotide sequence as set forth in SEQ ID NO.:1 or SEQ ID NO.: 6. The isolated nucleic acid may comprise at least one, preferably at least two, more preferably at least three, of the nucleotide sequences as set forth in SEQ ID NO.: 11, SEQ ID NO.: 12, SEQ ID NO.: 13. The isolated nucleic acid may comprise at least one, preferably at least two, more preferably at least three, of the nucleotide sequences as set forth in SEQ ID NO: 14, SEQ ID NO.: 15, and SEQ ID NO.: 16.

In a further aspect of the invention is provided an expression vector, or a host cell comprising said expression vector, said expression vector comprising the nucleic acid as hereindescribed.

### BRIEF DESCRIPTION OF THE DRAWINGS

FIGS. 1a and 1b show the cytotoxicity effect of serial diluted alpha-hemolysin on A549 cells. A549 is a human adenocarcinomic alveolar basal epithelial cell line.

FIG. 2a shows the toxin neutralization activity of the CAN24G4 antibody in A549 cells with alpha-hemolysin at a concentration of 5 μg/mL.

FIG. 2b shows the toxin neutralization activity of the CAN24G5 antibody in A549 cells with alpha-hemolysin at a concentration of 5 μg/mL.

FIG. 3 shows the toxin neutralization activity of the CAN24G4 and CAN24G5 antibodies combination in 1:1 ratio in A549 cells with alpha-hemolysin at a concentration of 5 μg/mL.

FIG. 4a shows the toxin neutralization activity of CAN24G4 antibody in A549 cells pre-incubated with alpha-hemolysin for 0.5, 1, 2, 3, and 4 hours prior the addition of CAN24G4.

FIG. 4b shows the toxin neutralization activity of CAN24G5 antibody in A549 cells pre-incubated with alpha-hemolysin for 0.5, 1, 2, 3, and 4 hours prior the addition of CAN24G5.

FIG. 5 shows the cytotoxic effect of S. aureus (ATCC 29213 and NCTC 8325) culture supernatants on A549 cells.

FIG. 6a shows the protection of A549 cells from S. aureus culture supernatants-induced cytotoxic effect by CAN24G4.

FIG. 6b shows the protection of A549 cells from S. aureus culture supernatants-induced cytotoxic effect by CAN24G5.

FIG. 7a shows rabbit red blood cell lysis upon exposure to alpha-hemolysin contained in supernatant from S. aureus NCTC 8325 strain.

FIG. 7b shows rabbit red blood cell lysis upon exposure to alpha-hemolysin contained in supernatant from S. aureus ATCC 29213 strain.

FIG. 8a shows the protective activity of CAN24G4 antibody in rabbit red blood cells from lysis upon exposure to alpha-hemolysin containing supernatants from S. aureus ATCC 29213 and NCTC 8325 strains.

FIG. 8b shows the protective activity of CAN24G5 antibody in rabbit red blood cells from lysis upon exposure to alpha-hemolysin containing supernatants from S. aureus ATCC 29213 and NCTC 8325 strains.

FIG. 9 shows Western blot analysis of alpha-hemolysin in supernatants from S. aureus strains ATCC 29213, NCTC 8325 and a commercial source of alpha-hemolysin detected with sheep polycolonal antibody to S. aureus alpha-hemolysin.

FIG. 9b shows Western blot analysis of alpha-hemolysin in S. aureus strains ATCC 29213, NCTC 8325 and a commercial source of alpha-hemolysin detected with mouse polycolonal antibody to S. aureus alpha-hemolysin.

FIG. 10 shows a Western blot analysis of alpha-hemolysin in supernatants from S. aureus strains ATCC 29213, NCTC 8325 and a commercial source of alpha-hemolysin detected with CAN24G4 antibody.

FIG. 11 shows a Western blot analysis of alpha-hemolysin in supernatants from S. aureus strains ATCC 29213, NCTC 8325 and a commercial source of alpha-hemolysin detected with CAN24G5 antibody.

FIG. 12 shows the inhibition of alpha toxin heptameric oligomerization (HLA) by CAN24G4 antibody in rabbit red blood cells upon exposure to alpha-toxin.

FIG. 13 shows the neutralization activity of CAN24G4 and CAN24G5 antibody in rabbit red blood cells and thus preventing the hemolysis with 1 ug/mL alpha-toxin. 50% neutralization titer (NT50%) for CAN24G4 and CAN24G5 are 0.1 and 0.5 ug/mL respectively.

FIG. 14 shows Western blot analysis of alpha-hemolysin in cultural supernatant of S. aureus strain NCTC 8325 along with its isogenic agr (agr is a well known global regulator in S. aureus which activates the alpha toxin production) and hla (alpha toxin) mutants. Commercially purified alpha-hemolysin was used as a positive control.

FIG. 15 shows the survival rate over time for CAN24G4 immunized mice challenged with a lethal dose of S. aureus in a bacteremia model, compared to sham-immunized and mice immunized with a GP-3E4 antibody control.

FIG. 16 shows photographs of lesions in CAN24G4 immunized mice challenged intradermally with alpha toxin, as compared to control mice, four days after challenge in a dermal necrosis model.

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference. As used in this specification and the appended claims, the singular forms “a”, “an” and “the” include plural references unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Vertebrate,” “mammal,” “subject,” “mammalian subject,” or “patient” are used interchangeably and refer to mammals such as human patients and non-human primates, as well as experimental animals such as rabbits, rats, and mice, cows, horses, goats, and other animals. Animals include all vertebrates, e.g., mammals and non-mammals, such as mice, sheep, dogs, cows, avian species, ducks, geese, pigs, chickens, amphibians, and reptiles.

The present invention generally relates to compositions and methods for the prevention or treatment of bacterial infection by S. aureus, in a vertebrate. Methods for inducing an immune response to S. aureus infection are provided. The methods provide administering an antibody or agent to subject in need thereof in an amount effective to reduce, eliminate, or prevent S. aureus bacterial infection or bacterial carriage.

Compositions and methods are provided for inducing an immune response to S. aureus hemolysin in a subject comprising administering to the subject a composition comprising an isolated polypeptide, such as S. aureus alpha-hemolysin antigens, and an adjuvant in an amount effective to induce the immune response in the subject. The method can be used for the generation of antibodies for use in passive immunization or as a component of a vaccine to prevent infection or relapse from infection by S. aureus.

The term “adjuvant” refers to an agent which acts in a nonspecific manner to increase an immune response to a particular antigen or combination of antigens, thus, for example, reducing the quantity of antigen necessary in any given composition and/or the frequency of injection necessary to generate an adequate immune response to the antigen of interest. See, e.g., A. C. Allison J. Reticuloendothel. Soc. (1979) 26:619-630. Such adjuvants are described further below. The term “pharmaceutically acceptable adjuvant” refers to an adjuvant that can be safely administered to a subject and is acceptable for pharmaceutical use.

“Bacterial carriage” is the process by which bacteria can thrive in a normal subject without causing the subject to get sick. Bacterial carriage is a very complex interaction of the environment, the host and the pathogen. Various factors dictate asymptomatic carriage versus disease. Therefore an aspect of the invention includes treating or preventing bacterial carriage.

“Treating” or “treatment” refers to either (i) the prevention of infection or reinfection, e.g., prophylaxis, or (ii) the reduction or elimination of symptoms of the disease of interest, e.g., therapy. “Treating” or “treatment” can refer to the administration of a composition comprising a polypeptide of interest, e.g., S. aureus alpha-hemolysin antigens or antibodies raised against these antigens. Treating a subject with the composition can prevent or reduce the risk of infection and/or induce an immune response to the polypeptide of interest. Treatment can be prophylactic (to prevent or delay the onset of the disease, or to prevent the manifestation of clinical or subclinical symptoms thereof) or therapeutic suppression or alleviation of symptoms after the manifestation of the disease.

Preventing” or “prevention” refers to prophylactic administration or vaccination with polypeptide or antibody compositions.

“Therapeutically-effective amount” or “an amount effective to reduce or eliminate bacterial infection” or “an effective amount” refers to an amount of polypeptide or antibody that is sufficient to prevent S. aureus bacterial infection or to alleviate (e.g., mitigate, decrease, reduce) at least one of the symptoms associated with S. aureus bacterial infection to reduce bacterial burden in blood or tissues or to induce an immune response to S. aureus alpha-hemolysin protein. It is not necessary that the administration of the composition eliminate the symptoms of S. aureus bacterial infection, as long as the benefits of administration of compound outweigh the detriments. Likewise, the terms “treat” and “treating” in reference to S. aureus bacterial infection, as used herein, are not intended to mean that the subject is necessarily cured of infection or that all clinical signs thereof are eliminated, only that some alleviation or improvement in the condition of the subject is effected by administration of the composition.

As used herein, the term “immune response” refers to the response of immune system cells to external or internal stimuli (e.g., antigen, cell surface receptors, cytokines, chemokines, and other cells) producing biochemical changes in the immune cells that result in immune cell migration, killing of target cells, phagocytosis, production of antibodies, other soluble effectors of the immune response, and the like.

“Protective immunity” or “protective immune response,” is intended to mean that the subject mounts an active immune response to a composition, such that upon subsequent exposure to S. aureus bacteria or bacterial challenge, the subject is able to combat the infection. Thus, a protective immune response will generally decrease the incidence of morbidity and mortality from subsequent exposure to S. aureus bacteria among subjects. A protective immune response may also generally decrease colonization by S. aureus bacteria in the subjects.

“Active immune response” refers to an immunogenic response of the subject to an antigen, e.g., S. aureus alpha-hemolysin antigens. In particular, this term is intended to mean any level of protection from subsequent exposure to S. aureus bacteria or antigens which is of some benefit in a population of subjects, whether in the form of decreased mortality, decreased symptoms, such as bloating or diarrhea, prevention of relapse, or the reduction of any other detrimental effect of the disease, and the like, regardless of whether the protection is partial or complete. An “active immune response” or “active immunity” is characterized by “participation of host tissues and cells after an encounter with the immunogen. It generally involves differentiation and proliferation of immunocompetent cells in lymphoreticular tissues, which lead to synthesis of antibody or the development cell-mediated reactivity, or both.” Herbert B. Herscowitz, “Immunophysiology: Cell Function and Cellular Interactions in Antibody Formation,” in Immunology: Basic Processes 117 (Joseph A. Bellanti ed., 1985). Alternatively stated, an active immune response is mounted by the host after exposure to immunogens by infection, or as in the present case, by administration of a composition. Active immunity can be contrasted with passive immunity, which is acquired through the “transfer of preformed substances (e.g., antibody, transfer factor, thymic graft, interleukin-2) from an actively immunized host to a non-immune host.”

“Passive immunity” refers generally to the transfer of active humoral immunity in the form of pre-made antibodies from one individual to another. Thus, passive immunity is a form of short-term immunization that can be achieved by the transfer of antibodies, which can be administered in several possible forms, for example, as human or animal blood plasma or serum, as pooled animal or human immunoglobulin for intravenous (IV) or intramuscular (IM) use, as high-titer animal or human immunoglobulin for IV or IM use from immunized subjects or from donors recovering from a disease, and as monoclonal antibodies. Passive transfer can be used prophylactically for the prevention of disease onset, as well as, in the treatment of several types of acute infection. Typically, immunity derived from passive immunization lasts for only a short period of time, and provides immediate protection, but the body does not develop memory, therefore the patient is at risk of being infected by the same pathogen later.

In some embodiments, once the S. aureus alpha-hemolysin antigen is overexpressed and purified, it is prepared as an immunogen for delivery to a host for eliciting an immune response. The host can be any animal known in the art that is useful in biotechnological screening assays and is capable of producing recoverable antibodies when administered an immunogen, such as but not limited to, rabbits, mice, rats, hamsters, goats, horses, monkeys, baboons, and humans. In one aspect, the host is transgenic and produces human antibodies, e.g., a mouse expressing the human antibody repertoire, thereby greatly facilitating the development of a human therapeutic.

As used herein, the term “antibody” refers to any immunoglobulin or intact molecule as well as to fragments thereof that bind to a specific epitope. Such antibodies include, but are not limited to polyclonal, monoclonal, chimeric, humanized, single chain, Fab, Fab′, F(ab)′ fragments and/or F(v) portions of the whole antibody and variants thereof. All isotypes are emcompassed by this term, including IgA, IgD, IgE, IgG, and IgM.

As used herein, the term “antibody fragment” refers specifically to an incomplete or isolated portion of the full sequence of the antibody which retains the antigen binding function of the parent antibody. Examples of antibody fragments include Fab, Fab′, F(ab′)2, and Fv fragments; diabodies; linear antibodies; single-chain antibody molecules; and multispecific antibodies formed from antibody fragments.

An intact “antibody” comprises two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind. Examples of antigen binding portions include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature, 341:544-546 (1989)), which consists of a VH domain; and (vi) an isolated complementarity determining region (CDR).

As used herein, the term “single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science, 242:423-426 (1988); and Huston et al., Proc Natl Acad Sci USA, 85:5879-5883 (1988)). Such single chain antibodies are included by reference to the term “antibody” fragments can be prepared by recombinant techniques or enzymatic or chemical cleavage of intact antibodies.

As used herein, the term “human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT App. Pub. Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins can be produced. See, Cabilly, U.S. Pat. No. 4,816,567, incorporated herein by reference in its entirety and for all purposes; and Queen et al., Proc Natl Acad Sci USA, 86:10029-10033 (1989).

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one aspect, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “antigen” refers to a substance that prompts the generation of antibodies and can cause an immune response. It can be used interchangeably in the present disclosure with the term “immunogen”. In the strict sense, immunogens are those substances that elicit a response from the immune system, whereas antigens are defined as substances that bind to specific antibodies. An antigen or fragment thereof can be a molecule (i.e., an epitope) that makes contact with a particular antibody. When a protein or a fragment of a protein is used to immunize a host animal, numerous regions of the protein can induce the production of antibodies (i.e., elicit the immune response), which bind specifically to the antigen (given regions or three-dimensional structures on the protein). The antigen can include, but is not limited to, S. aureus alpha-hemolysin proteins and fragments thereof.

As used herein, the term “humanized antibody,” refers to at least one antibody molecule in which the amino acid sequence in the non-antigen binding regions and/or the antigen-binding regions has been altered so that the antibody more closely resembles a human antibody, and still retains its original binding ability.

In addition, techniques developed for the production of “chimeric antibodies” (Morrison, et al., Proc Natl Acad Sci, 81:6851-6855 (1984), incorporated herein by reference in their entirety) by splicing the genes from a mouse antibody molecule of appropriate antigen specificity together with genes from a human antibody molecule of appropriate biological activity can be used. For example, the genes from a mouse antibody molecule specific for an autoinducer can be spliced together with genes from a human antibody molecule of appropriate biological activity. A chimeric antibody is a molecule in which different portions are derived from different animal species, such as those having a variable region derived from a murine mAb and a human immunoglobulin constant region.

In addition, techniques have been developed for the production of humanized antibodies (see, e.g., U.S. Pat. No. 5,585,089 and U.S. Pat. No. 5,225,539, which are incorporated herein by reference in their entirety). An immunoglobulin light or heavy chain variable region consists of a “framework” region interrupted by three hypervariable regions, referred to as complementarity determining regions (CDRs). Briefly, humanized antibodies are antibody molecules from non-human species having one or more CDRs from the non-human species and a framework region from a human immunoglobulin molecule.

Alternatively, techniques described for the production of single chain antibodies can be adapted to produce single chain antibodies against an immunogenic conjugate of the present disclosure. Single chain antibodies are formed by linking the heavy and light chain fragments of the Fv region via an amino acid bridge, resulting in a single chain polypeptide. Fab and F(ab′)2 portions of antibody molecules can be prepared by the proteolytic reaction of papain and pepsin, respectively, on substantially intact antibody molecules by methods that are well-known. See e.g., U.S. Pat. No. 4,342,566. Fab′ antibody molecule portions are also well-known and are produced from F(ab′)2 portions followed by reduction of the disulfide bonds linking the two heavy chain portions as with mercaptoethanol, and followed by alkylation of the resulting protein mercaptan with a reagent such as iodoacetamide.

After the host is immunized and allowed to elicit an immune response to the immunogen, a screening assay can be performed to determine if the desired antibodies are being produced. Such assays may include assaying the antibodies of interest to confirm their specificity and affinity and to determine whether those antibodies cross-react with other proteins.

The terms “specific binding” or “specifically binding” refer to the interaction between the antigen and their corresponding antibodies. The interaction is dependent upon the presence of a particular structure of the protein recognized by the binding molecule (i.e., the antigen or epitope). In order for binding to be specific, it should involve antibody binding of the epitope(s) of interest and not background antigens.

Once the antibodies are produced, they are assayed to confirm that they are specific for the antigen of interest and to determine whether they exhibit any cross reactivity with other antigens. One method of conducting such assays is a sera screen assay as described in U.S. App. Pub. No. 2004/0126829, the contents of which are hereby expressly incorporated herein by reference. However, other methods of assaying for quality control are within the skill of a person of ordinary skill in the art and therefore are also within the scope of the present disclosure.

Antibodies, or antigen-binding fragments, variants or derivatives thereof of the present disclosure can also be described or specified in terms of their binding affinity to an antigen. The affinity of an antibody for an antigen can be determined experimentally using any suitable method. (See, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer.

Antibody kinetics, along with other physical properties and immunogenicity often dictate their utility. Higher affinity antibody will be able to either bind to its target ligand faster (determined by the Association rate constant, k_{A}), or stay bound longer (determined by the Dissociation rate constant, k_{d}) or some of both properties.

Antibody affinity can be defined as the strength of the reaction between a single antigenic determinant and a single combining site on the antibody. It is the sum of the attractive and repulsive forces operating between the antigenic determinant and the combining site of the antibody and can only be measured quantitatively for monoclonal and not for polyclonal abs due to avidity effects.

While the epitope is critical to a highly potent mAb, in some cases there can be a correlation between potency of a monoclonal antibody and the affinity depending factors such as the epitope targeted, tissue distribution, antigen form, concentration, and bio-activity (Zuckier et al., 2000). Furthermore, the clinical use of an antibody having high affinity as well as potency can also translate into higher efficacy in vivo (Li et al., 2002; Zhu et al., 2003). The value in this is that mAbs with very high affinity may be able to be used at much lower doses in order to achieve the desired clinical effects, such as protection, recovery etc. This is important because lower dosing may allow for formulation into more convenient administration and smaller injection volumes, which would translate into a lower cost of goods for manufacturing.

A mAb is considered to be of high affinity has a KD in the nanomolar range (10⁻⁸ to 10⁻⁹) (Griffiths et al., 1994; de Haard et al., 1999) and occasionally in the sub-nanomolar range (Vaughan et al., 1996; Rathanaswami et al., 2005) (also called high picomolar). There are a number of technologies available to measure the affinity of an antigen-antibody interaction known to those skilled in the art. These include technologies such as: Surface Plasmon resonance (SPR) (eg. GE's Biacore) (Jonsson et al., 1991) Bio-Layer interferometry (eg. ForteBio's QKe system) (Abdiche et al., 2008); Solution based kinetic exclusion assay (eg. Sapidyne's KinExA) ¹⁰, and others are used to measure equilibrium constants (Rathanaswami et al., 2005; Rich et al., 2009). All these methods have inherent limitations such as the need for purified antigens, inability to measure cell borne antigens, low range, or need to fix the antigen depending upon the system (Rich et al., 2009).

The term “isolated protein,” “isolated polypeptide,” or “isolated peptide” is a protein, polypeptide or peptide that by virtue of its origin or source of derivation (1) is not associated with naturally associated components that accompany it in its native state, (2) is free of other proteins from the same species, (3) is expressed by a cell from a different species, or (4) does not occur in nature. Thus, a peptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The terms “polypeptide”, “protein”, “peptide,” “antigen,” or “antibody” within the meaning of the present invention, includes variants, analogs, orthologs, homologs and derivatives, and fragments thereof that exhibit a biological activity, generally in the context of being able to induce an immune response in a subject, or bind an antigen in the case of an antibody.

The polypeptides of the invention include an amino acid sequence derived from S. aureus alpha-hemolysin antibodies or fragments thereof, corresponding to the amino acid sequence of a naturally occurring protein or corresponding to variant protein, i.e., the amino acid sequence of the naturally occurring protein in which a small number of amino acids have been substituted, added, or deleted but which retains essentially the same immunological properties. In addition, such derived portion can be further modified by amino acids, especially at the N- and C-terminal ends to allow the polypeptide or fragment to be conformationally constrained and/or to allow coupling to an immunogenic carrier after appropriate chemistry has been carried out. The polypeptides of the present invention encompass functionally active variant polypeptides derived from the amino acid sequence of S. aureus alpha-hemolysin antibodies in which amino acids have been deleted, inserted, or substituted without essentially detracting from the immunological properties thereof, i.e. such functionally active variant polypeptides retain a similar or identical antibody activity and specificity.

Functionally active variants comprise naturally occurring functionally active variants such as allelic variants and species variants and non-naturally occurring functionally active variants that can be produced by, for example, mutagenesis techniques or by direct synthesis.

For the purpose of the present invention, it should be considered that several antibodies or fragments thereof of the invention may be used in combination. All types of possible combinations can be envisioned.

### EXAMPLES

### Example 1

### Hybridoma Fusion

A classical hybridoma fusion was performed. Mice received their first immunization with S. aureus alpha-hemolysin using Complete Freund's Adjuvant (CFA) and two subsequent boosters on days 28 and 48 with alpha-hemolysin and Incomplete Freund's Adjuvant (IFA). A trial bleed was performed at day 55 and the serum was tested to check for IgG titres of anti-alpha-hemolysin antibody. If IgG titres were high enough fusions began. If not, mice received two more boosts of alpha hemolysin with IFA and a second trial bleed was taken. Fusions were performed using 2 mice at a time. Mice were given a final “push” intraperitoneally (i.p.) with alpha-hemolysin in PBS three days prior to the fusion.

The day of the fusion, mice were sacrificed and their spleens removed. Splenocytes were washed from the spleen using a syringe and needle and collected in a 50 ml tube for fusion with myeloma cells, an immortal tumor cell line used as fusion partners, grown in the presence of 8-azaguanine, a toxic nucleotide analog which blocks the salvage pathway. Cells grown in the presence of 8-aza survive only by incurring defective mutations in the hypoxanthine-guanine phosphoribosyl transferase (HGPRT) gene. B cells were fused with the myeloma cells using Polyethylene Glycol (PEG) 1500. Fused cells were mixed into semi-solid agarose with drug selection and plated out into petri dishes. HAT media containing Hypoxanthine, Aminopterin, and Thymidine was used for drug selection. Aminopterin is a drug which inhibits the de novo pathway for nucleotide metabolism which is absolutely required for survival/cell growth in myeloma lines defective in HGPRT, usually within 24-48 hours un fused myelomas begin to die.

### Example 2

### Hybridoma Selection

During the hybridoma selection, ELISA screening was performed and multiple stages of the cell line growth while being expanded from individual wells of 96 well plates and into T-flasks. The cell lines were frozen down in a cryopreservative freezer media for long term storage. During this process spent cell supernatant was used to determine the secreted mAb isotyps for a given clonal cell line. The decision to move a clone to the next stage of selection was based on its diluted strength of reactivity to alpha-hemolysin using an ELISA and its survival, therefore the number of cell lines decreased throughout the selection procedure. Multiple fusions were performed from the mice immunized with alpha-hemolysin. The cell lines which passed through to the final stages of screening were grown up in tissue culture for production of the mAb in larger quantity and subsequently purified using standard protein A chromatography and characterized. Two such promising clones were purified for characterization; the antibodies produced therefrom were identified as “CAN 24G4” and “CAN 24G5”.

### Example 3

### Toxin Cytotoxicity Assay and Toxin Neutralization Assay

Preparation of the A549 Cells.

A549 cells, a human adenocarcinomic alveolar basal epithelial cell line was used for in vitro neutralization assays. Adherent cells are harvested from flasks using standard trypsin digestion. The cells were washed and treated with 3 ml of trypsin for 5 minutes at 37° C.5% CO₂. Following this, 7 ml of complete growth medium was added and the cells were aspirated by gentle pipetting. The viable cells were determined by trypsan blue exclusion. Cells were seeded into two 96-well flat bottom culture plates (DMEM/F12 media) at 1.5×10⁴ cells/well. The plates were incubated at 37° C./5% CO₂ while the toxin and MAb dilution were completed.

Measuring the Cytotoxicity of the Alpha-Hemolysin.

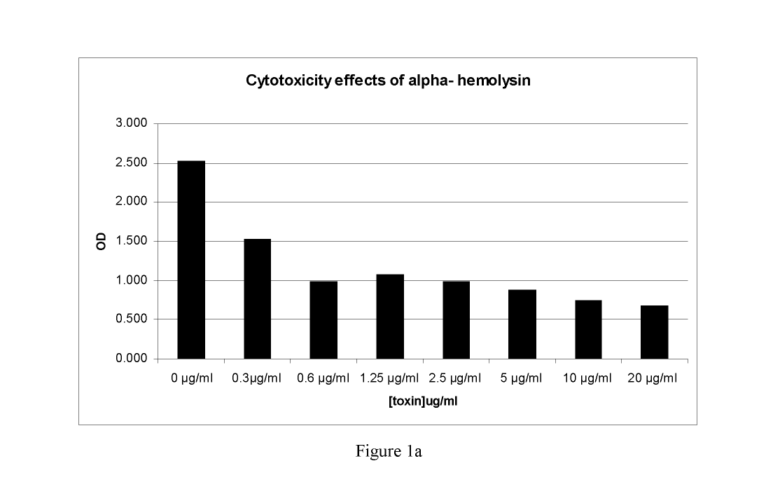

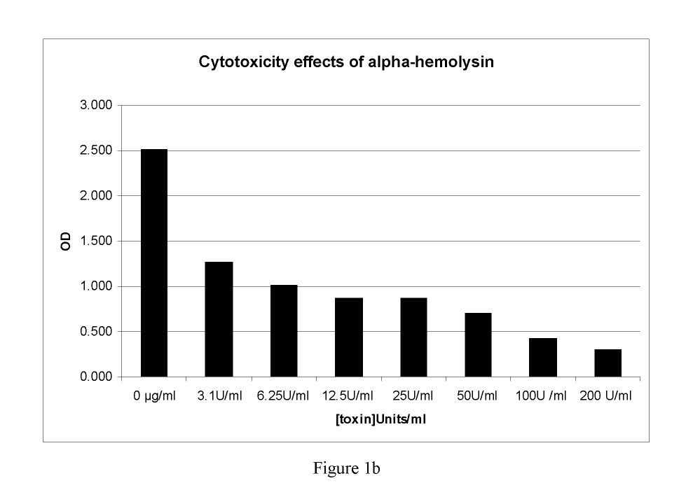

Cytotoxic effects of the alpha-hemolysin were measured by adding 0, 0.3, 0.6, 1.25, 2.5, 5, 10, and 20 μg per ml of alpha-hemolysin to wells each containing 1.5×10⁴ cells. The experiment was repeated, using 3.1, 6.25, 12.5, 25, 50, 100, and 200 Units per ml of alpha-hemolysin. The toxin-containing wells were incubated at 37° C./5% CO₂ overnight. The toxicity effects were measured by WST-1 assay, which monitors the conversion of the tetrazolium salt to the formazan dye by metabolically active cells and quantified by measuring the relative absorbance at approximately 440 nm wavelength. The cytotoxicity of the alpha-hemolysin on the A549 cells was measured and shown, in chart form, in FIGS. 1(a) and (b). FIG. 1(a) shows the cytotoxicity effects of alpha-hemolysin (measured as optical density of the well at 440 nm), at varying concentrations of alpha-hemolysin, measured as μg/mL. FIG. 1(b) shows the same cytotoxicity effects of alpha-hemolysin, at varying unit concentrations of the toxin.

Preparation of the Antibodies.

For the toxin neutralization assay, each of the two monoclonal antibodies, CAN24G4 and CAN24G5 were diluted to 200 m/ml. 200 μl of diluted antibody was added to 96-well plate, in triplicate. Wells were serially diluted, with 50 μl from each dilution was transferred to appropriate wells of 96 well plates.

Addition of the Alpha-Hemolysin.

The toxin was diluted to 20 μg/mL and 50 μL was added to each well containing the antibodies described above. 50 uL of assay medium was added to control wells. Plates with wells containing antibodies and toxin, as well as control wells containing antibodies alone, or toxin alone, or neither antibody or toxin, were incubated at 37° C./5% CO₂ for 1 hour. 50 μL of the toxin/antibody mixture or controls was transferred to wells containing 1.5×10⁴ A549 cells, as described above, and the plates were incubated at 37° C./5% CO₂ overnight.

Detection of Neutralization by Antibodies.

The cell viability was monitored by the WST-1 assay, as described above, and higher OD values correlate with neutralization of the toxin effects by treatment with the antibodies. 10 μL of WST-1 reagent was added into each well of the plate after the toxin antibody incubation. The plate was incubated for 1 hour at 37° C./5% CO₂. The absorbance was measured at 440 nm wavelength.

Effect of Antibodies.

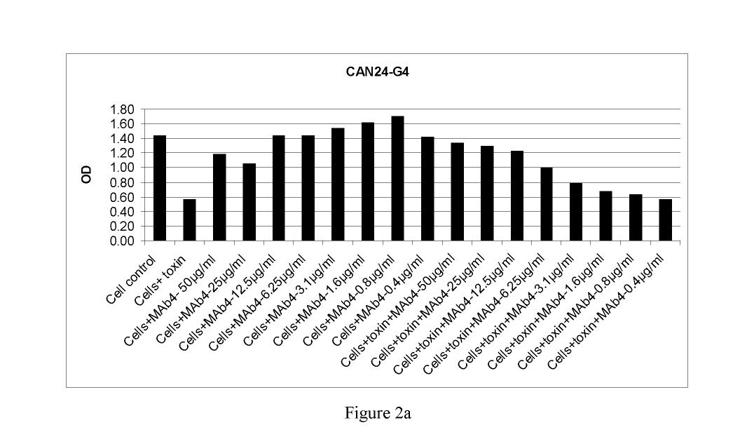

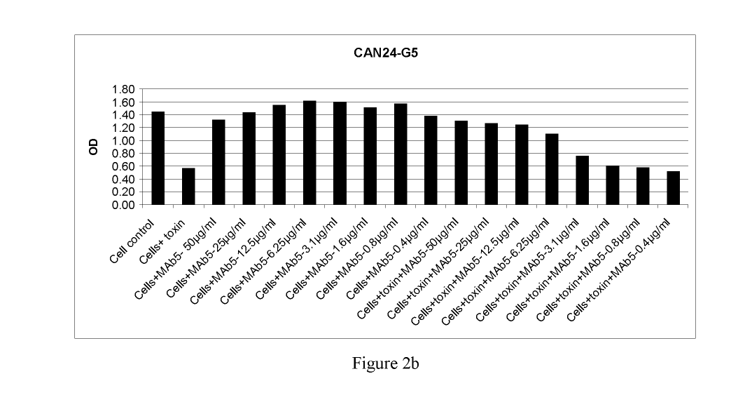

The optical density of each well was measured, as a measure of cell viability, and the results were charted as FIG. 2a (for antibody CAN24G4) and 2b (for antibody CAN24G5). FIG. 2 charts show the optical density of control cells (containing neither toxin nor antibody, “Cell control”), as compared to cells containing 5 μg/mL alpha-hemolysin toxin (“Cells+toxin”). Wells containing 50, 25, 12.5, 6.25, 3.1, 1.6, 0.8 and 0.4 μg/mL of the antibody, and not containing toxin, showed little to no cell death, as would be expected. Wells containing both toxin and antibody (50, 25, 12.5 and 6.25 μg/mL) showed a neutralization of alpha-hemolysin, based on the protection of the cells from the cytotoxic effect of the toxin, in what appeared to be an antibody concentration-dependent manner, as compared to the control wells containing cells and toxin alone. Note that wells containing less than 6.25 μg/ml of antibody (3.1, 1.6, 0.8 and 0.4 μg/ml) did not show significant effect in this model.

Effect of Combination of Antibodies.

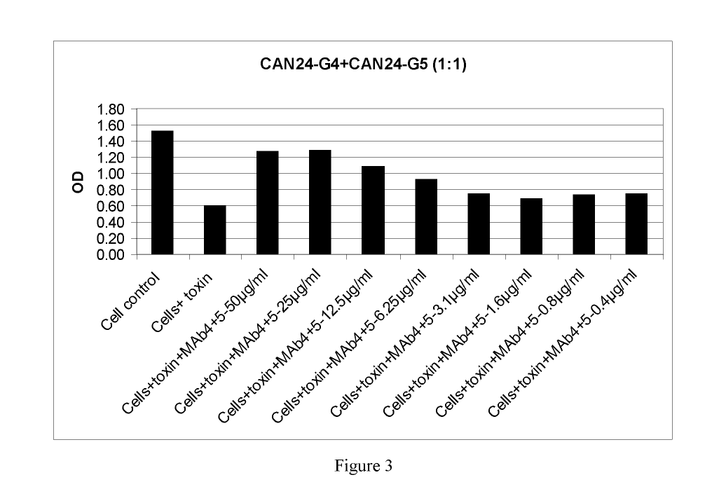

The experiment was repeated, this time with both the CAN24G4 and the CAN24G5 antibodies, in a 1:1 ratio, in each of the treatment groups. Thus, cells were incubated with either no antibody and no toxin (Cell control); toxin only (5 μg/mL) (Cells+toxin), or a combination of toxin (5 μg/mL) and both CAN24G4 and CAN24G5 antibodies in a 1:1 ratio, at a range of concentrations (as described). Results were graphed and shown as FIG. 3. As seen in those results, wells containing both antibodies neutralized alpha-hemolysin toxicity, and at low concentrations were even better at neutralizing alpha-hemolysin toxicity than either antibody alone, even at concentrations as low as 0.4 μg/mL. Comparing these results to those shown in FIGS. 2a and b, it appears that the combination of the two antibodies may have synergistic results over either antibody alone.

Time Course Experiments.

The experiment was repeated again, this time delaying introduction of the monoclonal antibody after the addition of the toxin to the cells. Cells were subjected to alpha-hemolysin toxin, and incubated at 37° C./5% CO₂ for varying times before the addition of the monoclonal antibody. Various concentrations of monoclonal antibody was added, and the cells were then incubated at 37° C./5% CO₂ overnight. The cell viability was measured by WST-1 assay, as described and absorbance was measured at 440 nm.

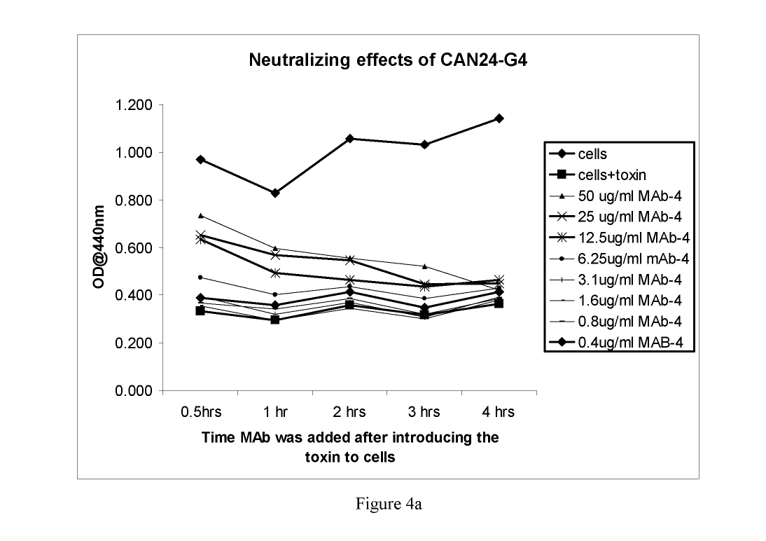

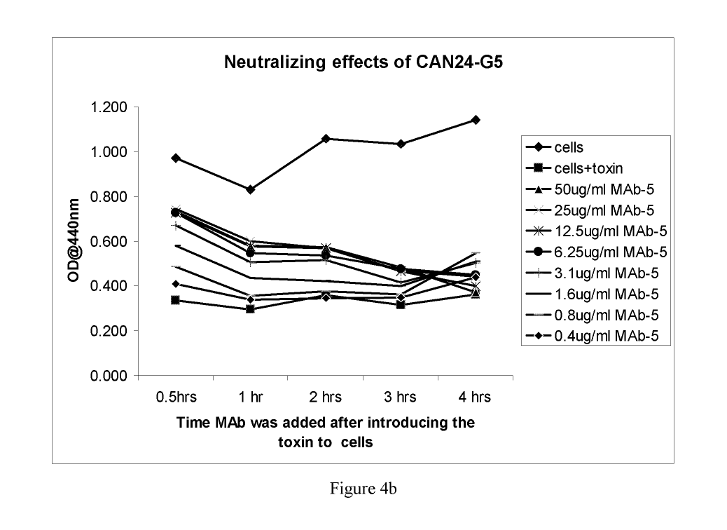

The neutralizing effects of CAN24G4 at varying times after exposure to toxin was shown in chart form as FIG. 4a. The effects of CAN24G5 antibody was shown in chart form as FIG. 4b. As can be seen, there was both a dose-dependency, and a time dependency to the neutralization effects of both antibodies, with higher concentrations of antibody, given sooner after subjection of the cells to toxin, having a better neutralizing effect. Both antibodies clearly showed neutralizing effects, even when administered 2-4 hours after subjecting the cells to toxin.

### Example 4

### Neutralization of Cytotoxic Effects of S. aureus Supernatants

Preparation of S. aureus Supernatants.

Staphylococcus aureus (S. aureus) samples (ATCC 29213, NCTC 8325) that were frozen in cryogenic beads were removed from the −80° C. freezer and four-way streaked onto a TSA plate. The plates were inverted and incubated at 37° C. for 24 hrs. The plates were checked for purity and a few colonies of the S. aureus were removed and placed into 3 mL TSB to achieve a McFarland standard of 1. A 1/15 dilution was prepared by adding 1 mL of the inoculum to 14 mL TSB (A P&G plate was prepared identically for each strain). For the supernatants, 75 μL of the 1/15 inoculum was added to 75 μL of media in each well of a 96 well plate. The lid was placed on the plate, the plate was sealed and the plate placed in a shaking incubator at 37° C. for 48 hours. The media was collected and filtered. A streak plate was conducted on each sample to ensure no bacterial growth was present. The samples were stored in the −80° C. freezer until use.

An ELISA capture was used to quantify the amount of alpha-hemolysin in the supernatant samples. The ELISA results showed 48.8 μg/mL of alpha-hemolysin in the ATCC 29213 supernatant, and 390.6 μg/mL in the NCTC 8325 supernatant.

Cytotoxic Effects of S. aureus Supernatants on A549 Cells.

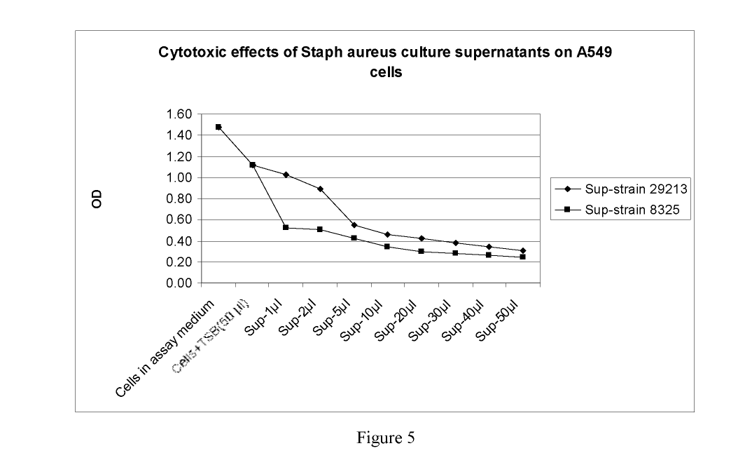

A549 cells were prepared in wells as described for Example 3, above. Varying amounts of each S. aureus supernatant was added to the cells; the cells were then incubated overnight at 37° C./5% CO₂. Cell viability was measured using the WST-1 assay, as described for Example 3, as optical density of the wells when measured at 440 nm. A chart summarizing the results is found as FIG. 5. As can be seen even as little as 1 μL of supernatant had toxic effect on the cells, with a dramatic toxicity seen at 5 μL and greater. This toxicity was found for both strains of S. aureus supernatant tested.

Toxin Neutralization Assay.

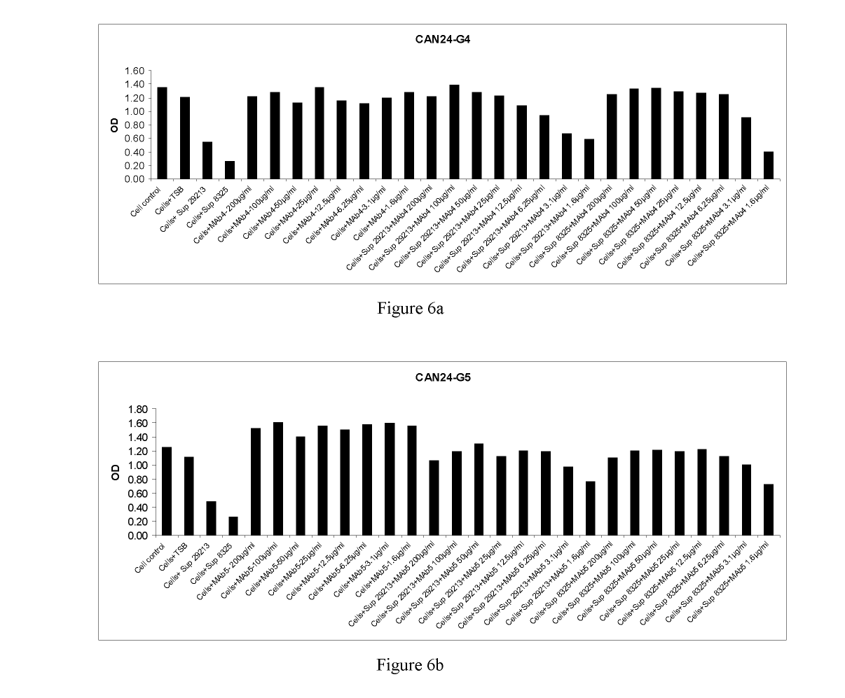

The assay of Example 3 was repeated, utilizing the 5 μL of each S. aureus culture supernatant described and characterized above, instead of pure alpha-hemolysin as the toxin. The results of this toxin neutralization assay, using A549 cells, were charted as FIGS. 6a and 6b, for antibodies CAN24G4 and CAN24G5, respectively. Shown are a Cell control (no toxin, no antibody); Cells+TSB (a TSB control with no toxin, no antibody); Cells+Supernatant 29213 (S. aureus ATCC 29213) and Cells+Supernatant 8325 (S. aureus NCTC 8325) (both positive controls, where cells were subjected to the two hereinbefore described toxins); Cells+MAb 4 or 5 (Cells+antibody, at varying concentrations, but no toxin); Cells+Supernatant 29213+MAb4 or 5 (cells+antibody+toxin, at varying concentrations of antibody); Cells+Supernatant 8325+MAb 4 or 5 (cells+antibody+toxin, at varying concentrations of antibody). As can be seen, both antibodies had excellent, dose-dependent, neutralization effects on the toxicity of both S. aureus supernatants. Note that higher Optical Density correlated with higher metabolic activity and cell viability, based on WST-1 assay as described in Example 3. The figures showed that CAN24G4 and CAN 24G5 antibodies both protected cells from toxic effect of S. aureus supernatants, in a range of doses.

### Example 5

### Red Blood Cell Hemolysis Assay

Preparation of Rabbit Red Blood Cells (rRBC). 10 mL of rabbit blood was mixed with 20 mL 0.9% NaCl; mixed gently by inversion and centrifuged at 2200 rpm for 5 minutes and supernatant removed. The rabbit red blood cells (rRBC) were washed in the same manner with 20 ml each of PBS, repeated three times. The washed rRBC were stored at 2-8° C. until use. 5% and 20% RBC suspensions were prepared as described: 20% suspension was made with 12 mL PBS+3 mL of washed rRBC; 5% suspension was made with 6 mL PBS+2 mL of rRBC at 20% suspension.

The S. aureus supernatants (ATCC 29213, NCTC 8325) as described above, were diluted with PBS to defined concentration.

Preparation of Antibodies and S. aureus Supernatants.

The monoclonal antibodies were prepared at 400 μg/mL. The monoclonal antibodies were 2-fold serially diluted using PBS in titer tubes. Each concentration of monoclonal antibody was mixed 1:1 with either PBS or S. aureus supernatants in 96 well plates. The MAb and S. aureus supernatant mixture was incubated for 1 hr at room temperature.

RBC Hemolysis by S. aureus Supernatants.

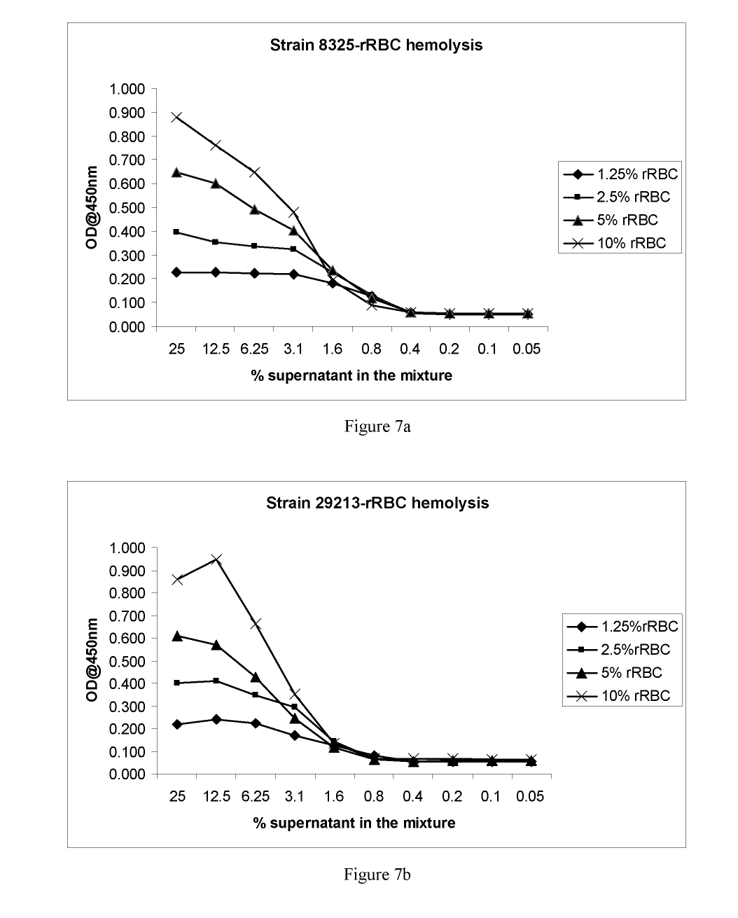

Washed rRBC, prepared as described above, were added at either 10%, 5%, 2.5% or 1.25% final concentration to each well of a U-bottom 96 well plate. S. aureus supernatants, as described above, were added to each well in varying amounts. The plate was incubated at room temperature for 1 hr. The plate was centrifuged at 2500 rpm for 5 minutes. 50 μL was removed and place on microplate for reading at 450 nm. Cell lysis was measured at the optical density of 450 nm, (the higher OD, the higher the level of cell death). FIG. 7a shows rRBC hemolysis due to the presence of supernatant from S. aureus strain NCTC 8325. As can be seen, the amount of hemolysis was concentration dependent on both the % of S. aureus supernatant and the concentration of rRBC. Similar results were shown in FIG. 7b for S. aureus strain ATCC 29213.

Antibody Protection of RBC Hemolysis.

50 μL of washed rRBC, prepared as described above, were added at 10% final concentration to each well of a U-bottom 96 well plate. 50 μl of the MAb/supernatants mixture was added to each well according to the plate layout (Table 1). For the S. aureus supernatants, the final concentration in each well was 25% of the full strength of the supernatants. For the MAbs, the concentrations were from 100 μg/mL to 0.78 μg/mL. The positive hemolysis control (Cells+1% Triton) was 50 μL of cells+50 μL of a 1% Triton X solution. The negative control (Cells+PBS) was 50 μL of cells+50 μL of PBS. The positive supernatants controls (Cells+ATCC29213; Cells+NCTC8325) were each 50 μL cells+50 μL diluted supernatants at 50% full strength. The plate was incubated at room temperature for 1 hr. The plate was centrifuged at 2500 rpm for 5 minutes. 50 μL was removed and optical density measured at 450 nm.

TABLE 1
Plate layout for antibody treatment of 10% rabbit red blood cells

| | 1 | 2 | 3 | 4 | 5 | 6 | 7 | 8 | 9 | 10 | 11 | 12 |
|---|---|---|---|---|---|---|---|---|---|---|---|---|
| A | Cells | Cells | 100 | 100 | 100 | 100 | 100 | 100 | 100 | 100 | 100 | Cells |
| B | + | + | 50 | 50 | 50 | 50 | 50 | 50 | 50 | 50 | 50 | + |
| C | PBS | ATCC | 25 | 25 | 25 | 25 | 25 | 25 | 25 | 25 | 25 | 1% |
| D | | 29213 | 13 | 13 | 13 | 13 | 13 | 13 | 13 | 13 | 13 | Triton |
| E | | Cells | 6.3 | 6.3 | 6.3 | 6.3 | 6.3 | 6.3 | 6.3 | 6.3 | 6.3 | |
| F | | + | 3.13 | 3.13 | 3.13 | 3.13 | 3.13 | 3.13 | 3.13 | 3.13 | 3.13 | |
| G | | NCTC | 1.56 | 1.56 | 1.56 | 1.56 | 1.56 | 1.56 | 1.56 | 1.56 | 1.56 | |
| H | | 8325 | 0.78 | 0.78 | 0.78 | 0.78 | 0.78 | 0.78 | 0.78 | 0.78 | 0.78 | |
| | | | Cells | Cells | Cells | Cells | Cells | Cells | Cells | Cells | Cells | |
| | | | + | + | + | + | + | + | + | + | + | |
| | | | MAb 3 | MAb 3 | MAb 3 | MAb 4 | MAb 4 | MAb 4 | MAb 5 | MAb 5 | MAb 5 | |
| | | | | + | + | | + | + | | + | + | |
| | | | | Sup 1 | Sup 2 | | Sup 1 | Sup 2 | | Sup 1 | Sup 2 | |

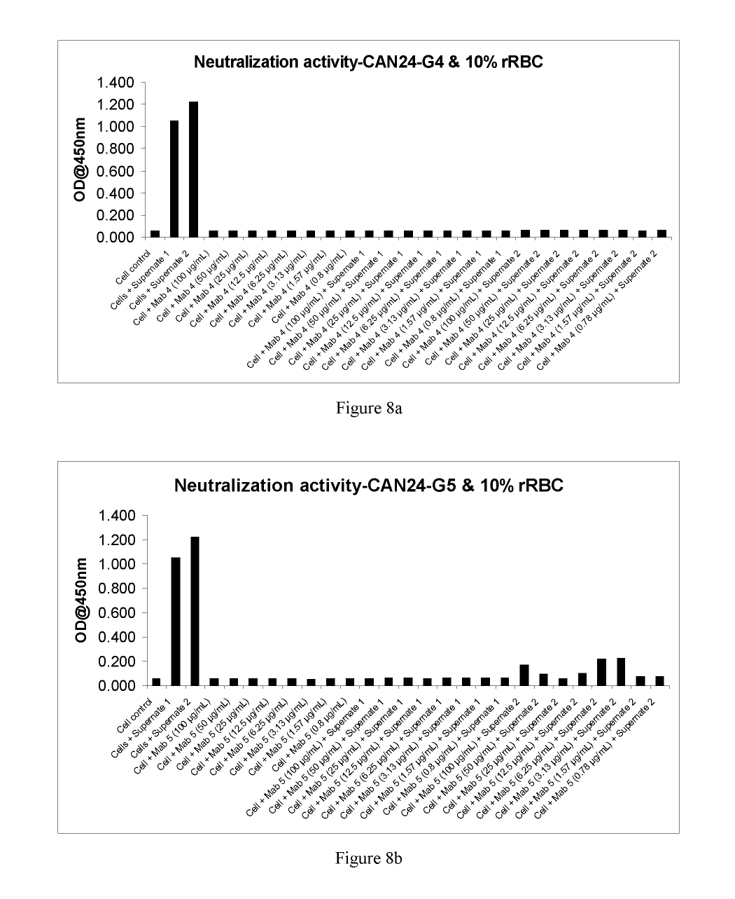

FIG. 8a shows the neutralization activity of CAN24G4 antibody, in a 10% rabbit Red Blood Cell mixture. As can be seen, both supernate 1 (supernatant from S. aureus strain 8325) and supernate 2 (supernatant from S. aureus strain 29213) caused significant hemolysis, which was prevented through the addition of the monoclonal antibody as low as 0.8 μg/ml. FIG. 8b shows similar results, for antibody CAN24G5. Both CAN24G4 and CAN24G5 were thus able to totally protect rabbit red blood cells from lysis induced by S. aureus supernatants in all concentrations tested.

### Example 6

### Confirmation of Antibody Alpha-Hemolysin Found in S. aureus Strains 29213 and 8325 Supernatants and Purified with Alpha-Hemolysin

Western Blot Analysis of Alpha-Hemolysin.

15 μg of S. aureus (ATCC29213 and NCTC8325) culture supernatants and 10 μg commercial alpha-hemolysin (Toxin Technology Inc.) were resolved by Electrophoresis, and proteins were transferred to nitrocellulose membrane. Mouse and sheep anti-Alpha-hemolysin polyclonal antibodies and Cangene monoclonal antibodies (Can24G4 and Can24G5) were used at a dilution of 1:1000 to probe the blots respectively before its development with SIGMAFAST™ BCIP®/NBT.

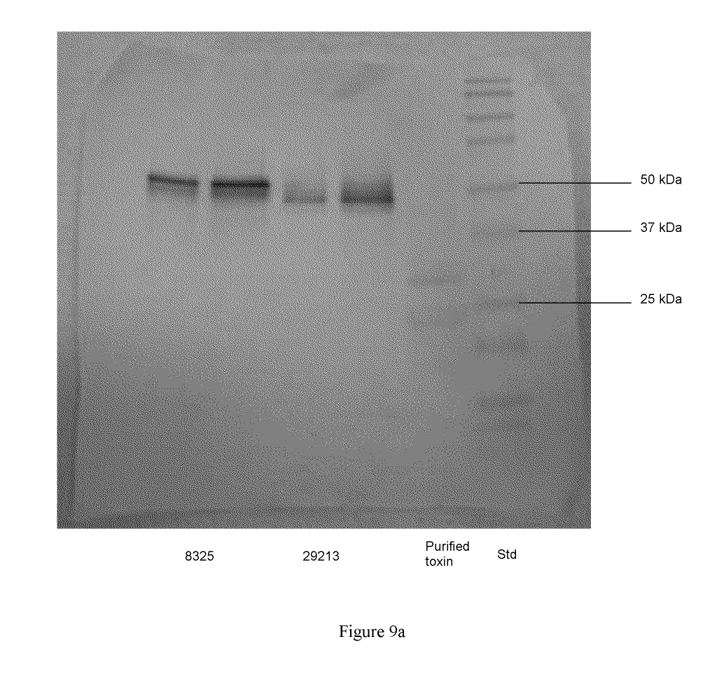

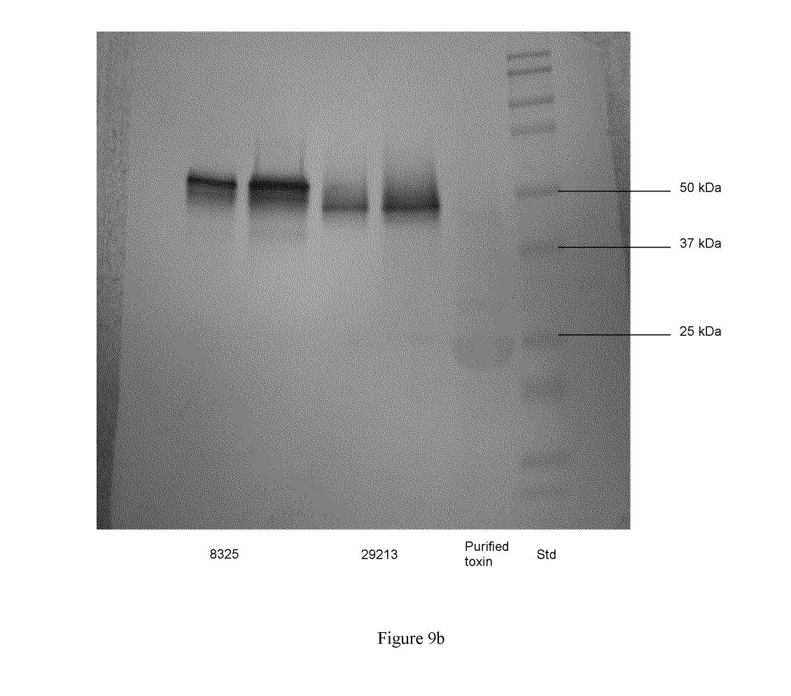

Western blot was performed to detect and compare alpha-hemolysin in S. aureus ATCC 29213 and NCTC 8325 supernatants with commercial toxin as shown in FIG. 9. Two polyclonal antibodies (sheep and mouse) to alpha-hemolysin were used to detect alpha-hemolysin and similar results were observed (FIGS. 9a and b, respectively). Antibody binding to the purified toxin produced a faint band in the expected range of 30 kDa. The faint bands were probably due to the lower concentration of toxin loaded into the gel. The protein bands that were detected in the S. aureus supernatants fell within the 50 kDa range indicating either alpha-hemolysin exists in a complex, either a dimer, or with another protein, in S. aureus supernatants. However, the results confirmed the presence of alpha-hemolysin in the supernatant from both S. aureus strains. FIG. 9a shows a Western blot analysis of S aureus strain 8325 supernatant (rows 1 and 2), S aureus strain 29213 supernatant (rows 3 and 4) and purified commercially available alpha-hemolysin (row 5), against a sheep polyclonal antibody specific for S. aureus alpha-hemolysin. FIG. 9b shows the same data, against a mouse polyclonal antibody to S. aureus.

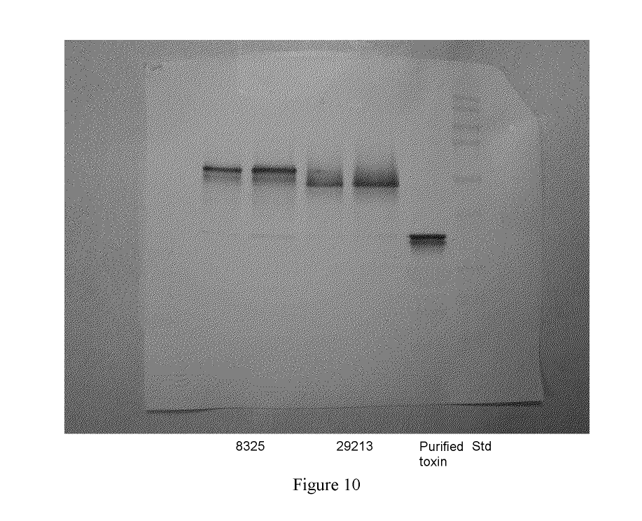

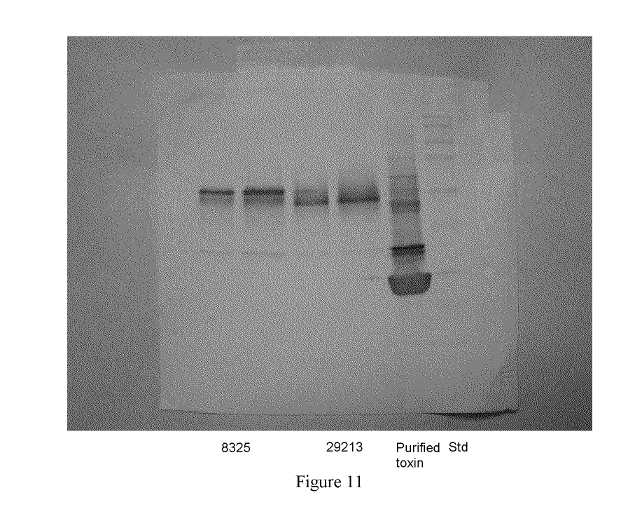

Two monoclonal antibodies (CAN24G4 and CAN24G5) were also used to confirm their binding to alpha-hemolysin by Western blot. Results showed that similar bands at 50 kDa range were detected by both monoclonals and compared to polyclonal antibodies; however, staining with CAN24G4 and CAN24G5 also illustrated faint bands at the expected 30 kDa range when alpha-hemolysin presented as monomer for both supernatants and a strong band for purified toxin. FIG. 10 shows the same data, detected with the CAN 24G4 antibody. FIG. 11 shows the same data, detected with the CAN24G5 antibody.

### Example 7

### Monoclonal Antibody Binding to Alpha-Hemolysin and Epitope Competition

Biacore assay was used to test the relative binding of two monoclonal antibodies: Can24G4 and Can25G5 to alpha-hemolysin. Alpha hemolysin was immobilized on a Biacore CM5 chip, the antibodies were tested for relative binding affinities as measured by Plasmon-surface resonance relative units (RU). To further evaluate the binding epitopes of these monoclonal antibodies, epitope competition assay was performed by measuring the binding of a second antibody, after the first antibody had bound, to monitor additional binding to different epitopes. Results showed that both monoclonal antibodies had approximately the same binding affinity for alpha-hemolysin of 126.6 RU and 105.2 RU for CAN 24G4 and CAN 24G5, respectively (Table 2). Affinity of both antibodies is found to be better than (lower than) 10e-8. The epitope competition assay did not show additive binding above background levels for each antibody. The overlay of CAN 24G4 and CAN 24G5 only induced 13.7% increased binding, which suggested that CAN 24G4 and CAN 24G5 likely recognize the same epitope (Table 3).

TABLE 2
Binding affinity of CAN24G4 and CAN25G5

| | Chemical/Reagent | Relative Response |
|---|---|---|
| Antibody | Number | (RU) |
| Abcam anti-hemolysin | 705.00107 | 91.9 |
| polyclonal | | |
| CAN 24G4 | RC.0158 | 126.6 |
| CAN 24G5 | RC.0159 | 105.2 |

TABLE 3
Epitope mapping of CAN24G4 and CAN25G5

| | Relative | | Relative | | |
|---|---|---|---|---|---|
| First | Response | Second | Response | % | |
| Antibody | (RU) | Antibody | (RU) | Increase | Result |
| CAN 24G5 | 183.6 | CAN24G5 | 14.1 | 7.7 | Background |
| | | | | | binding |
| CAN 24G5 | 183.6 | CAN24G4 | 25.1 | 13.7 | Likely |
| | | | | | same epitope |
| CAN 24G4 | 166.7 | CAN24G4 | 7.0 | 4.2 | Background |
| | | | | | binding |

Plasmon Resonance (Biacore) Assay for Determination of Binding Kinetics of mAb Can24G4 for Alpha Hemolysin.

To eliminate avidity effects due to the bivalency of IgG, the analytical strategy entailed capture of the monoclonal antibody by its Fc region and subsequent flow of analyte over the chip surface. A CM5 sensor chip (BR-1000-14) was used with a Mouse Antibody Capture Kit (BR-1008-38) to generate a sensor chip with approximately 6000 RUs of immobilized anti-human IgG. Scouting experiments were performed for to identify the appropriate amount of ligand (anti-alpha-hemolysin mAb) for capture and the appropriate concentration of analyte. These conditions are indicated in Table 4. Running buffer for all experiments was HBS-EP+ (BR-1006-69).

Results: Table 5 shows the binding kinetics of the CAN24G4 in an affinity measurement by BIAcore analysis. This antibody had affinity in the low-nanomolar range (9.4 nM) showing that this MAb was able to form a tight association with alpha-hemolysin, a parameter essential for toxin neutralization.

TABLE 4
parameters used for the BIAcore assay
for the affinity measurement of CAN24G4

| | | Association | Dissociation | Concentrations |
|---|---|---|---|---|
| | Ligand | time | time | of analyte tested |
| mAb | capture | (sec) | (sec) | (ng/mL) |
| CAN24G-4 | 180 s of | 180 | 600 | 1000, 333, |
| | 0.2 μg/mL | | | 111, 37, 12 |

TABLE 5
binding kinetics of mAb Can24G4 for alpha hemolysin
(BIAcore assay for the affinity measurement)

| mAb | ka (1/Ms) | kd (1/s) | KD (M) |
|---|---|---|---|
| CAN24G-4 | 4.3 × 10⁵ | 4.0 × 10⁻³ | \| 9.4 × 10⁻⁹ |

### Example 8

### Oligomerization Inhibition Assay

Rabbit erythrocytes (RRBC) from Colorado serum co. were washed two times with PBS, spun, and the resultant pellet was re-suspended in PBS to final concentration of 10% RRBC (wt/vol.). 50 uL of CAN24G4 mAb at 40 ug/ml was added to 50 uL of 4 ug/mL of alpha toxin for 10 minutes at room temperature for neutralization. Then 100 uL of 10% RRBC was added to the mixture and then incubated at 37° C. for 30 minutes. Final concentration of antibody and alpha-hemolysin in the reaction mixtures were 10 ug/mL and 1 ug/mL respectively. Two controls samples were processed in parallel, one with the same concentration of alpha-hemolysin but without CAN24G4; the second containing PBS and 10% RRBC only. After incubation, samples were centrifuged at 3,700 RPM for 10 minutes at 4° C. The supernatants were removed and pellets were washed in 500 uL of PBS to remove excess alpha-hemolysin from the reaction mixture. Pellets were re-suspended in 100 uL SDS loading dye. 15 uL of the samples were run on 4-15% SDS PAGE. Proteins were then transferred from the SDS PAGE to nitrocellulose membrane. Western blots were performed by using a sheep anti-alpha hemolysin polyclonal (Toxin Tech) as primary antibody and detected by Goat anti-sheep-AP conjugate as secondary antibody.

Results:

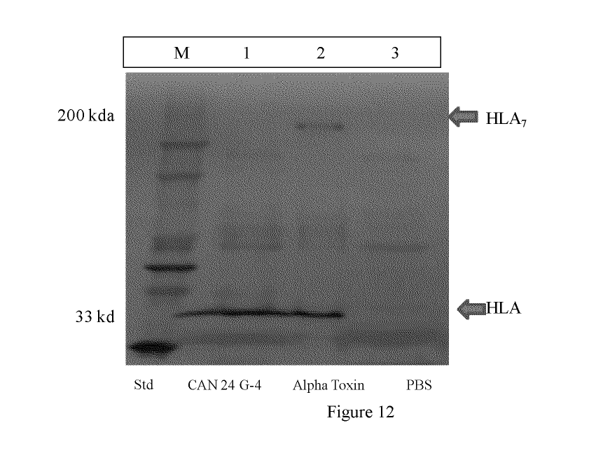

CAN24G4 mAb mediated inhibition of alpha toxin oligomerization in presence of rabbit erythrocytes. As shown in the FIG. 12, 500 ng (50 uL of 10 ug/ml) CAN24G4 completely blocked the heptameric (HLA₇) band pointed by arrow at approx. 200 kd (Lane 1). Positive control sample with same concentration of alpha-hemolysin gave a very prominent HLA₇ band (lane 2). Both of these samples showed the monomeric band (HLA) of 33 kd size indicating that CAN24G4 did not block the binding HLA in RRBC receptor but only blocked the oligomerization step. Lane 3 is the RRBC control in absence of alpha toxin. Data shown in FIG. 12 indicated that CAN24G4 inhibited formation of Hla heptamers suggesting this as a potential mechanism of action.

### Example 9

### Alpha-Hemolysin Toxin Neutralization Assay

50 uL of 4 ug/mL of alpha-hemolysin toxin (Hla) (Toxin Tech) was added to 50 μL of serial dilutions of CAN24G4 or CAN24G5 in PBS in a 96 well ELISA plate (Nunc). The mixture was incubated at room temperatures for 10 minutes for neutralization. 100 uL of 2% RRBC in PBS was then added to the reaction mix to a final toxin concentration to 1 ug/mL. The reaction mixtures were incubated for 30 minutes at 37° C. followed by centrifugation at 3,700 rpm for 10 minutes. 100 uL of the supernatants were transferred in new ELISA plate without disturbing the pellet and the absorbance at 416 nm was measured. 50% neutralization titers (NT50) were calculated by plotting the mAb concentration against OD416 nm in a full 4-PL curve in Softmax (Molecule device) by plotting.

Results:

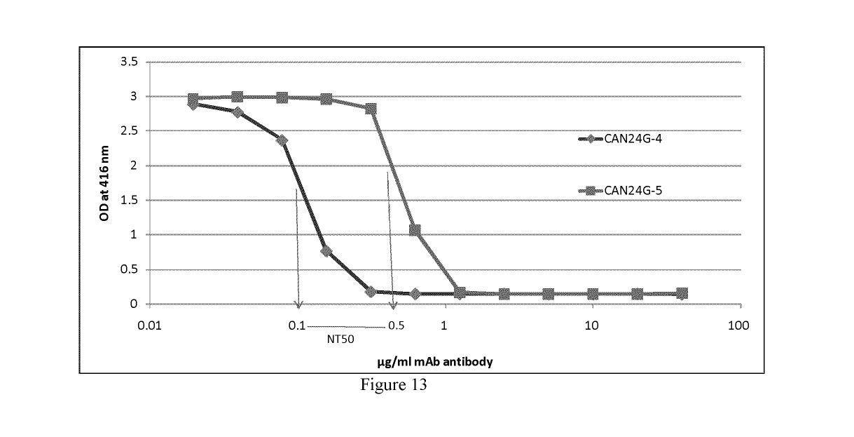

FIG. 13 shows the alpha-hemolysin toxin neutralization titer (NT50) of CAN24G4 and CAN24G5 mAbs. CAN24G-4 showed low NT50 titer of 0.1 ug/mL when tested with 1 ug/mL alpha toxin (Toxin Tech) in 2% RRBC. This value was 13 fold less than the sheep anti-alpha hemolysin pAb (Toxin Tech) tested (data not shown).

### Example 10

### Western Blot Analysis

Western blot was performed to detect alpha-hemolysin in S. aureus NCTC 8325 supernatants along with its relevant isogenic mutants by using CAN24G4. Bacterial cultural were prepared from a single colony of bacteria from BHI plate. Overnight bacterial cultures were prepared in 5 ml BHI broth at 37° C. shaker incubator (300 RPM). The cultures were then centrifuged and concentrated 5× with Amicon 3K cutoff filter. 15 ul of the concentrated supernatants were loaded in Bio-rad SDS PAGE. The Commercial alpha toxin from LIST BIOLOGICAL Inc. was used as a positive control. MAb CAN24G4 (20 ug/ml) was used as primary antibody against alpha toxin and then detected by conjugate (Goat anti-mouse AP antibody).

Results:

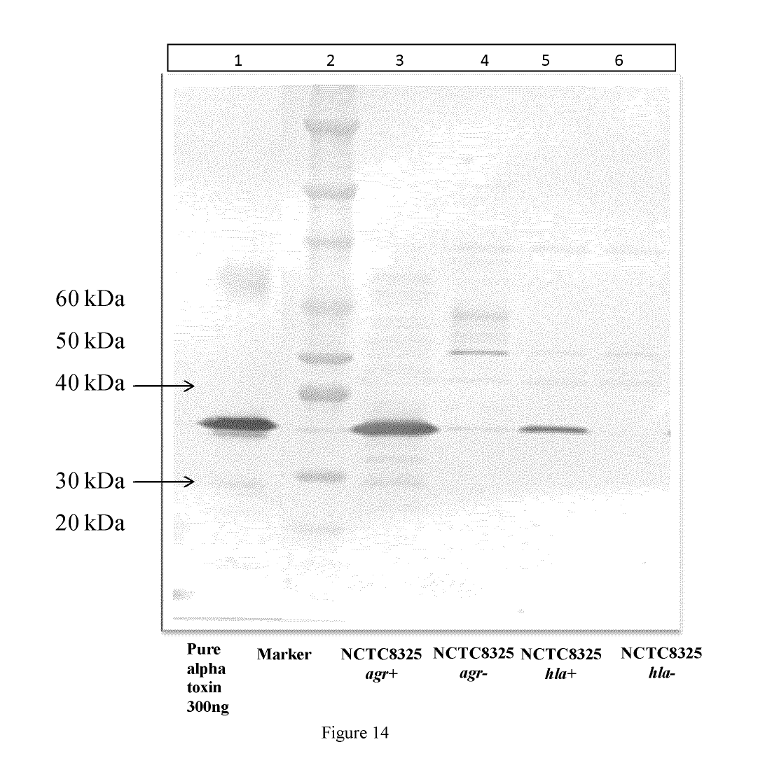

This MAb clearly detected the 34 kDa monomeric band of commercially purified alpha toxin (FIG. 14 lane 1) as well as alpha hemolysin toxin present in wild type S. aureus 8325 supernatant (lanes 3 and 5). This antibody was alpha hemolysin toxin specific as culture supernatant from isogenic agr and hla mutant (lanes 4 and 6) did not show the alpha toxin band.

### Example 11

### Protective Efficacy of CAN24G4-1 mAb in Bacteremia and Dermal Necrosis Models

The efficacy of CAN24-G4 monoclonal antibody was tested in mice models for bacteremia and dermal necrosis. Mice were immunized with 500 μg of antibody, 24 hours prior to bacterial challenge via three different routes, to test for efficacy against bacteremia and dermal necrosis. A control antibody (a non-specific monoclonal antibody GP-3E4, isotype IgG1, generated against a filo virus protein), as well as a mock immunization using PBS, were used as controls.

### Bacteremia

Mice were challenged with a lethal dose of S. aureus USA300 in 500 μl PBS along with 3% Hog mucin and were monitored daily for mortality and morbidity (i.e. lethargy, hunched posture, ruffled fur) during the course of challenge. Weight checks were performed daily.

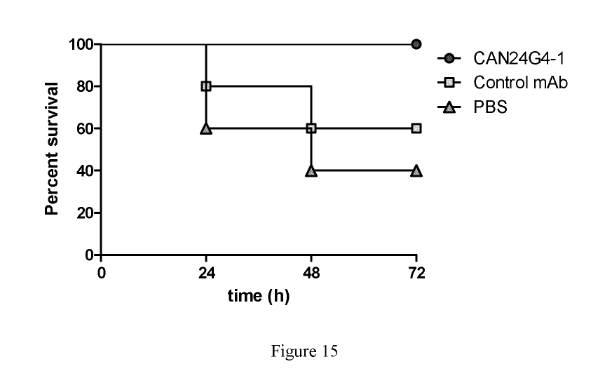

Results: As shown in FIG. 15, 100% of CAN24G4 immunized mice survived S. aureus challenge over a time period of 72 hours, whereas control mice only exhibited a 40% (PBS) or 60% (control antibody) survival rate over the same period. This shows excellent potential for CAN24G4 efficacy against bacteremia.

### Dermal Necrosis

Mice were challenged intradermally with 5 μg of wild type alpha toxin. Mice were observed for 4 days, lesion sizes were recorded and pictures of lesions were taken at t=24 h, 48 h and 72 h. Lesion became visible 48 h post inoculation and aggravated and peaked at t=4 days.

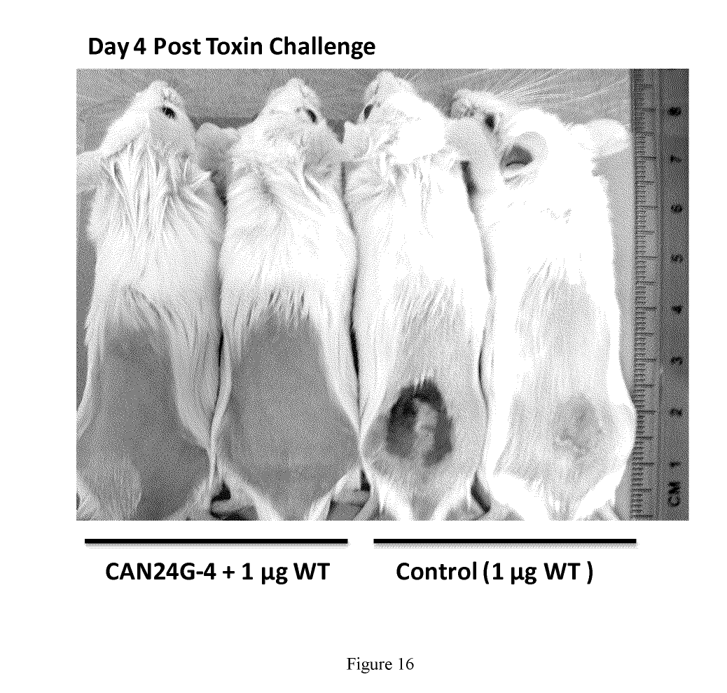

Results: As shown in FIG. 16, control mice exhibited much larger lesions at 96 hours (4 days) as compared to the CAN24G4-immunized mice.

### Example 12

### Sequencing of CAN24G4

The CAN24G4 monoclonal antibody was sequenced. The Heavy chain variable region was found to have the following nucleotide sequence (SEQ ID. No. 1):

gaggttcacttacagcagtctggggcagagcttgtgaagcctggggcctc
agtcaggttgtcctgcacaggttctggcttagacattaaagacacctata
ttcactgggtgaagaagaggcctgaacagggcctggagtggattggaagg
attgatcctgcgaatggtattactaaatatgacccgaagttccagggcaa
ggccactgtaacagcagacacatcctccaacacagcctacctgcagttca
gcagcctcacatcagaggacagtgccgtctactactgttcgagtgagtac
tatccttatcctatggactactggggtcaaggaacctcagtcaccgtctc
ctca

which translated into the following amino acid sequence (SEQ ID NO. 2):

EVHLQQSGAELVKPGASVRLSCTGSGLDIKDTYIHWVKKRPEQGLEWIGR
IDPANGITKYDPKFQGKATVTADTSSNTAYLQFSSLTSEDSAVYYCSSEY
YPYPMDYWGQGTSVTVSS.

Note that the start of CDR3 is not a typical CAR amino acid sequence, but rather, a CSS sequence. Thus, the amino acid and nucleotide CDR sequences of the heavy chain were, respectively, found to be:

CDR1: SEQ ID NO.: 3:
GLDIKDTY
CDR1: SEQ ID NO.: 11:
ggc tta gac att aaa gac acc tat
CDR2: SEQ ID NO.: 4:
I D P A N G I T
CDR2: SEQ ID NO: 12:
att gat cct gcg aat ggt att act
CDR3: SEQ ID NO.: 5:
S S E Y Y P Y P M D Y
CDR3: SEQ ID NO: 13:
tcg agt gag tac tat cct tat cct atg gac tac

The variable region of the Kappa light chain was found to have the following nucleotide sequence (SEQ ID NO. 6):

gacatcaagatgacccagcctccatcttccatgtatgcatctctaggaga
gagagtcactgtcacttgtaaggcgagtcaggacattagtagttttttaa
cctggttccaacagaaaccagggaaatctcctaagatcctgatctatcgt
gcaaacagagtggtagatggggtcccatcaaggttcagtggcagtggatc
tggccacgattattctctcaccatcagcagcctggagtctgaagatatgg
gaatttattattgtctacaatatgatgagtttccgtggacgttcggtgga
ggcaccaagctggagatcaaa

which translated into the following amino acid sequence (SEQ ID NO. 7):

DIKMTQPPSSMYASLGERVTVTCKASQDISSFLTWFQQKPGKSPKILIYR
ANRVVDGVPSRFSGSGSGEIDYSLTISSLESEDMGIYYCLQYDEFPWTFG
GGTKLEIK.

Thus, the CDR regions of the light chain were found to be:

CDR1: SEQ ID NO.: 8:
Q D I S S F
CDR1: SEQ ID NO.: 14:
cag gac att agt agt ttt
CDR2: SEQ ID NO.: 9:
R A N
CDR2: SEQ ID NO.: 15:
cgt gca aac
CDR3: SEQ ID NO.: 10:
L Q Y D E F P W T
CDR3: SEQ ID NO.: 16:
cta caa tat gat gag ttt ccg tgg acg

When specific aspects of the invention have been described and illustrated, such aspects should be considered illustrative of the invention only and not as limiting the invention as construed in accordance with the accompanying claims.

All publications and patent applications cited in this specification are herein incorporated by reference in their entirety for all purposes as if each individual publication or patent application were specifically and individually indicated to be incorporated by references for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1-64. (canceled)

65. An isolated monoclonal antibody, or antigen-binding portion thereof, comprising a heavy chain variable region (HCVR) and a light chain variable region (LCVR), wherein the HCVR comprises an amino acid sequence about 80%, 90%, 95% or 100% identical to SEQ ID NO: 2, and wherein the LCVR comprises an amino acid sequence about 80%, 90%, 95% or 100% identical to SEQ ID NO: 7.

66. The isolated monoclonal antibody, or antigen-binding portion thereof, of claim 65 wherein the HCVR comprises an amino acid sequence of SEQ ID NO: 2, and wherein the LCVR comprises an amino acid sequence of SEQ ID NO: 7.

67. The isolated antibody, or antigen binding portion thereof, of claim 65, wherein the HCVR and LCVR each comprise three complementarity determining regions (CDRs), CDR1, CDR2, CDR3, wherein the HCVR CDRs, CDR1, CDR2, CDR3, comprise amino acid sequences SEQ ID NOs: 3, 4 and 5, respectively, and wherein the LCVR CDRs, CDR1, CDR2, CDR3, comprise amino acid sequences SEQ ID NOs: 8, 9 and 10, respectively.

68. The isolated monoclonal antibody, or antigen-binding portion thereof, of claim 65, wherein the heavy chain variable region CDRs, CDR1, CDR2, CDR3, comprise amino acid sequences SEQ ID NOs: 3, 4 and 5, respectively.

69. The isolated monoclonal antibody, or antigen-binding portion thereof, of claim 65, wherein the light chain variable region CDRs, CDR1, CDR2, CDR3, comprise SEQ ID NOs: 8, 9 and 10, respectively.

70. The isolated monoclonal antibody, or antigen-binding portion thereof, of claim 65, comprising one CDR region, wherein the one CDR region comprises an amino acid sequence SEQ ID NO: 5.

71. The isolated monoclonal antibody, or antigen-binding portion thereof, of claim 65, wherein the isolated monoclonal antibody, or antigen-binding portion thereof, binds to S. Aureus alpha-hemolysin.

72. The isolated monoclonal antibody, or antigen-binding portion thereof, of claim 71, wherein the heavy chain variable region CDRs, CDR1, CDR2, CDR3, comprise amino acid sequences SEQ ID NOs: 3, 4 and 5, respectively.

73. The isolated monoclonal antibody, or antigen-binding portion thereof, of claim 71, wherein the light chain variable region CDRs, CDR1, CDR2, CDR3, comprise SEQ ID NOs: 8, 9 and 10, respectively.

74. A composition comprising the antibody or antigen-binding portion thereof of claim 65 and at least one pharmaceutically acceptable adjuvant.

75. A composition comprising the antibody or antigen-binding portion thereof of claims 65 and a pharmaceutically acceptable carrier.

76. The composition of claim 74 further comprising an antibiotic.

77. The composition of claim 74 further comprising an antibody.

78. A method of reducing or preventing S. Aureus infection comprising administering to a subject an effective amount of the antibody or antigen-binding portion thereof of claim 65.

79. A method of preventing or treating bactereamia comprising administering to a subject an effective amount of the antibody or antigen-binding portion thereof of claim 65.

80. A method of preventing or treating dermal necrosis comprising administering to a subject an effective amount of the antibody or antigen-binding portion thereof of claim 65.

81. The method of claims 78, 79, and 80, wherein the antibody or antigen-binding portion thereof, is administered intravenously, subcutaneously, intramuscularly or transdermally.

82. A method of reducing, preventing or treating any one of S. Aureus infection, bacteremia or dermal necrosis comprising administering to a subject an effective amount of the antibody or antigen-binding portion thereof of any one of claims 65-73.

83. A method of reducing, preventing or treating any one of S. Aureus infection, bacteremia or dermal necrosis comprising administering to a subject an effective amount of the isolated monoclonal antibody, or antigen-binding portion thereof, comprising a heavy chain variable region (HCVR) and a light chain variable region (LCVR), wherein the HCVR comprises an amino acid sequence about 80%, 90%, 95% or 100% identical to SEQ ID NO: 2, and wherein the LCVR comprises an amino acid sequence about 80%, 90%, 95% or 100% identical to SEQ ID NO: 7.

84. A method of reducing, preventing or treating any one of S. Aureus infection, bacteremia or dermal necrosis comprising administering to a subject an effective amount of the isolated monoclonal antibody, or antigen-binding portion thereof, of claim 83 wherein the HCVR comprises an amino acid sequence of SEQ ID NO:2 and wherein the LCVR comprises an amino acid sequence of SEQ ID NO: 7.

85. A method of reducing, preventing or treating any one of S. Aureus infection, bacteremia or dermal necrosis comprising administering to a subject an effective amount of the isolated monoclonal antibody, or antigen-binding portion thereof, of claim 83 wherein the HCVR and LCVR each comprise three complementarity determining regions (CDRs), CDR1, CDR2, CDR3, wherein the HCVR CDRs, CDR1, CDR2, CDR3, comprise amino acid sequences SEQ ID NOs: 3, 4 and 5, respectively, and wherein the LCVR CDRs, CDR1, CDR2, CDR3, comprise amino acid sequences SEQ ID NOs: 8, 9 and 10, respectively.

86. An isolated nucleic acid encoding the antibody or antigen-binding portion thereof, of claim 65, comprising a nucleic acid sequence with about 80%, 90%, 95% or 100% identity to SEQ ID NO: 11, SEQ ID NO: 12, SEQ ID NO: 13. SEQ ID NO: 14, SEQ ID NO: 15 and SEQ ID NO: 16.

87. An isolated nucleic acid encoding the antibody or antigen-binding portion thereof, of claim 65, comprising a nucleic acid sequence with about 80%, 90%. 95% or 100% identity to SEQ ID NO: 1 and SEQ ID NO: 6.

88. An expression vector comprising the nucleic acid of claim 86.

89. A host cell comprising the nucleic acid of claim 86.
